(19) United States
(12) Patent Application Publication
(10) Pub. No.: US 2010/0041606 A1
(43) Pub. Date: Feb. 18, 2010

(54) Methods of Use of Eggshell Polypeptides

(76) Inventor: Ilan Elias, Philadelphia, PA (US)

Correspondence Address: CANTOR COLBURN, LLP, 20 Church Street, 22nd Floor, Hartford, CT, 06103, US

(21) Appl. No.: 12/500,818

(22) Filed: Jul. 10, 2009

Related U.S. Application Data
(60) Provisional application No. 61/079,619, filed on Jul. 10, 2008.

(51) Int. Cl. A61K 38/38; C12N 5/071; A61P 19/08; A61K 38/18; A61K 38/17

(52) U.S. Cl. 514/12; 435/375

(57) ABSTRACT

Identified herein is an active component of chicken eggshells, which is shown ex-vivo studies to have a stimulating effect on bone building cells (osteoblasts). The substance is a polypeptide mixture extracted from eggshells. It has been discovered herein that a composition comprising eggshell polypeptides stimulates osteoblasts, and has osteoinductive/osteogenic properties, hematopoietic properties, and cartilage formation or differentiation properties. The eggshell polypeptide extracts can be locally or systemically administered.

### CROSS-REFERENCE TO RELATED APPLICATIONS

This application claims the benefit of U.S. Provisional Patent Application Ser. No. 61/079,619, filed Jul. 10, 2008, which is incorporated by reference herein in its entirety.

### BACKGROUND

Throughout life, old bone is continuously removed by bone-resorbing osteoclasts and replaced with new bone, which is formed by osteoblasts. This cycle is called the bone-remodeling cycle and is normally highly regulated, that is, the functioning of osteoclasts and osteoblasts is linked such that in a steady state the same amount of bone is formed as is resorbed.

The bone-remodeling cycle occurs at particular areas on the surfaces of bones. Osteoclasts which are formed from appropriate precursor cells within bones resorb portions of bone; new bone is then generated by osteoblast activity. Osteoblasts synthesize the collagenous precursors of bone matrix and also regulate its mineralization. The dynamic activity of osteoblasts in the bone remodeling cycle to meet the requirements of skeletal growth and matrix and also regulate its maintenance and mechanical function is thought to be influenced by various factors, such as hormones, growth factors, physical activity and other stimuli. Osteoblasts are thought to have receptors for parathyroid hormone and estrogen. There is a metabolic synergism between osteoclasts and osteoblasts. Osteoclasts adhere to the surface of bone undergoing resorption and are thought to be activated by a signal from osteoblasts. Osteoblasts, however, are also activated by a signal (e.g., released Ca) from osteoclasts. It is therefore important that both counterparts are active, in order to stimulate each other and to produce new bone. When treating an osteoporosis patient with bisphosphonates, for example, the patient's osteoclasts are diminished. It is uncertain as how active the osteoblasts will be in the long-term when receiving less signal from the remaining osteoclasts. For the human body it is essential to use both active osteoblast and active osteoclast to have new bone formation.

Irregularities in one or more stages of the bone-remodeling cycle (e.g., where the balance between bone formation and resorption is lost) can lead to bone remodeling disorders, or metabolic bone diseases such as osteoporosis and Paget's disease. Some of these diseases are caused by over-activity of one half of the bone-remodeling cycle compared with the other, such as by osteoclasts or osteoblasts. In osteoporosis, for example, there is a relative decrease of osteoblast activity, which may cause a reduction in bone density and mass. Osteoporosis is the most common of the metabolic bone diseases and may be either a primary disease or may be secondary to another disease or other diseases. Osteoporosis is characterized generally by a loss of bone density. Thinning and weakening of the bones leads to increased fracturing from minimal trauma.

Eggshells are a biological material comprising about 95-98% calcium carbonate, 1-2% trace minerals including Mg and others, and about 1-2% organic compounds such as proteins and collagens. Previously, commercially available chicken eggshell preparations, also referred to as Putamen Ovi (PO) or Biomin, have been used as oral medications to treat osteoporosis. (U.S. Pat. Nos. 6,344,217 and 7,011,853 by Ruepp; U.S. Pat. No. 5,045,323). These preparations undergo a heat processing (autoclaving, dry heat). While processing eggshells with heat the organic compounds of these preparations are diminished or even totally destroyed. However, autoclaved eggshell powder has been shown to have a stimulating effect on bone cells, however, it is unknown which substance within the native eggshell acts as the active agent and is responsible for stimulating effect. What is needed is the identification of the components of eggshells that are responsible for the bone cell stimulating effects and the use of these components in the treatment of bone disorders, bone injuries, and other conditions responsive to stimulation of osteoblast activity.

### BRIEF SUMMARY

In one embodiment, method of stimulating osteoblast activity comprises contacting osteoblasts with a composition comprising a polypeptide extract from eggshells, wherein the polypeptide extract is isolated from a hard eggshell tissue, a soft eggshell tissue, or a combination thereof.

In another embodiment, a method of stimulating osteogenic and/or osteoinductive activity in an individual in need thereof comprises administering to the individual a composition comprising a polypeptide extract from eggshells, wherein the polypeptide extract is isolated from a hard eggshell tissue, a soft eggshell tissue, or a combination thereof.

In yet another embodiment, a method of stimulating hematopoesis in an individual in need thereof comprises administering to the individual a composition comprising a polypeptide extract from eggshells, wherein the polypeptide extract is isolated from a hard eggshell tissue, a soft eggshell tissue, or a combination thereof.

In another embodiment, a method of stimulating cartilage formation and/or differentiation in an individual in need thereof comprises administering to the individual a composition comprising a polypeptide extract from eggshells, wherein the polypeptide extract is isolated from a hard eggshell tissue, a soft eggshell tissue, or a combination thereof.

In a further embodiment, a method of stimulating fibroblasts in an individual in need thereof comprises administering to the individual a composition comprising a polypeptide extract from eggshells, wherein the polypeptide extract is isolated from a hard eggshell tissue, a soft eggshell tissue, or a combination thereof.

Further included herein is the product of the process of treating hard eggshell tissue with a 1 to 25 wt % SDS and optionally 1 to 60% vol/vol acetic acid.

Yet further included is the product of the process of treating soft eggshell tissue with a solution comprising 2 to 10 M urea and having a pH of 7.0-9.0.

### BRIEF DESCRIPTION OF THE DRAWINGS

Referring now to the drawings wherein like

FIG. 1 is a schematic of exemplary purification of hard and soft eggshell polypeptides and the uses thereof.

FIG. 2 shows osteoblast proliferation with: bar 1—Control (no additions); bar 2—PO-I (Autoclaved at 137° C.); bar 3—PO-II (Standard autoclaving); bar 4—HA−Hard Eggshell Polypeptides 0.5 g/l [HA=Hyaluronic Acid]; bar 5—Hard Eggshell-Polypeptides (low concentration 0.005 g/l); bar 6—Hard Eggshell-Polypeptides (high concentration 0.5 g/l).

FIG. 3 shows osteoblast proliferation with: bar 1—Control (no additions); bar 2—PO-I (Autoclaved at 137° C.); bar 3—PO-II (Standard autoclaving); bar 4—HA−Hard Eggshell Polypeptides 0.5 g/l [HA=Hyaluronic Acid]; bar 5—Hard Eggshell-Polypeptides (low concentration 0.005 g/l); bar 6—Hard Eggshell-Polypeptides (high concentration 0.5 g/l); bar 7—PO-O (Autoclaved at 113° C.).

FIG. 4 shows the number of cells after 5 days in culture determined by the coulter counter system for three different protein samples A, B, C, B+C.

FIG. 5 shows the medium cell sizes of the osteoblast cells after 5 days in culture with MM1f-medium as control and media supplemented with protein samples A, B, C, B+C.

FIG. 6 shows a histogram of the medium cell size dependent on the medium supplemented with the protein samples A, B, C, B+C and MM1f as a control after 5 days in culture.

FIG. 7 shows a histogram of cell proliferation. Cells/image of primary bovine osteoblast-like cells cultured in media supplemented with protein samples A, B, C, B+C and MM1f as a control.

FIG. 8 shown the concentration of calcium in g/L after 4 weeks dependent on the used culture-medium, MM1f as control, medium with protein sample A and medium with protein sample B.

The above-described and other features will be appreciated and understood by those skilled in the art from the following detailed description, drawings, and appended claims.

### DETAILED DESCRIPTION

Identified herein is the active component of chicken eggshells, which is shown in in-vitro studies to have a stimulating effect on bone building cells (osteoblasts). The substance is one or more polypeptides extracted from eggshells. It has been discovered herein that a composition comprising eggshell polypeptide(s) stimulates osteoblasts, and thus has osteoinductive and/or osteogenic properties, hematopoietic properties, and cartilage formation and/or differentiation properties. As used herein, a polypeptide extract from eggshells is an extract from the eggshell hard shell tissue, soft shell tissue, or both, wherein the extract comprises greater than 95 wt %, specifically greater than 99.5 wt % eggshell polypeptides. In one embodiment, the polypeptide extract is a demineralized extract. After extraction, the polypeptide preparation contains minerals (mineral salts) such as calcium acetate. After extraction, the solution can be totally (95 wt %-99.5 wt %) purified using ultrafiltration to demineralize (desalting) the extract. After the demineralization the extract can be lyophilized and is then pure.

In one embodiment, the eggshells are chicken eggshells, that is, eggshells from Gallus gallus. In other embodiments, the eggshells are from goose (Anser anser), duck (Anas platyrhynchos) or ostrich (Struthio camelus).

In another embodiment, the eggshells are fresh eggshells. Fresh eggshells are defined herein as shells from eggs that are not treated/processed with heat, e.g., boiled, steam heated, or autoclaved. This feature distinguishes the present eggshell preparations from prior art preparations in which heat/steam are used to process the eggshells. Without being held to theory, it is believed that commercially available eggshell preparations (e.g. puamen ovi, Biomin) have a much lower polypeptide concentration than extracts from fresh eggshells. In other embodiments, the eggshells are from boiled, steam/heated. Fresh eggshells can be either from non-fertilized or from fertilized eggs.

It has been unexpectedly discovered that a composition comprising eggshell polypeptides has osteoinductive/osteogenic properties, that is, stimulates osteogenesis. In one embodiment, the composition comprising eggshell polypeptides is administered to an individual in need of treatment for a bone disorder such as osteoporosis, osteopenia, osteogenesis imperfecta, Paget's disease, bone fractures and the like.

It has further been unexpectedly discovered that a composition comprising eggshell polypeptides has hematopoietic properties, that is, stimulates hematopoiesis. In one embodiment, the composition comprising eggshell polypeptides is administered to an individual in need of treatment for anemia and related bone marrow and blood disorders.

It has further been unexpectedly discovered that a composition comprising eggshell polypeptides has cartilage formation and/or differentiation properties. In one embodiment, the composition comprising eggshell polypeptides is administered to an individual in need of repair of cartilage.

In one embodiment, the composition comprising eggshell polypeptides is in the form of a powder, a paste, a solution for injection, or a solution or paste for intraosseous injection or local application such as for use as a bone graft or bone stimulating agent, in Kyphoplasty, Vertiboplasty, into fracture site, into bone or joint defects. The foregoing compositions are useful for repair of bone and/or cartilage at sites of bone or cartilage injury or defect. The eggshell polypeptides are administered optionally in combination with a collagen sponge, a hyaluronan (hyaluronic acid) gel, calcium carbonate, (β)-tri calcium phosphate (TCP), calcium phosphate (Hydroxyapatite), osteoconductive degradable materials such as polymers (Polylactic, Polyglycolic Acids), a growth factor, or a combination comprising one or more of the foregoing agents. Exemplary growth factors include Bone Morphogenetic Proteins (BMPs), Transforming growth factor (TGFs), Fibroblast Growth factors (FGFs), Insulin-like growth factors (IGFs), Platelet-derived growth factors (PDGFs), and combinations comprising one or more of the foregoing growth factors.

In another embodiment, a composition comprising eggshell polypeptides is administered systemically, that is, in the form of an oral, nasal or injectable composition. Compositions for oral administration include tablets, capsules, and soft caps, for example. Systemic administration is suitable for the treatment of osteoporosis, osteopenia, and bone diseases such as Paget's disease, Osteogenesis Imperfecta, osteomalacia, osteopetrosis, Osgood-Schlatter disease, Algodystrophy, Reflex-Sympathetic-Dystrophy syndrome (Complex Regional Pain syndrome), transient osteoporosis, avascular necrosis, osteonecrosis, osteochondral lesions, osteolytic lesions, bone tumors and bone fractures. The eggshell polypeptides are optionally administered in combination with calcium, magnesium, phosphorus, fluoride, bisphosphonates, estrogens, vitamins (e.g. Vitamin A, D, E, K, C, B) Parathyroid hormone (PTH), trace minerals (e.g. zinc, manganese), soy flavonoids or a combination comprising one or more of the foregoing agents.

In one embodiment, systemic administration is suitable for the treatment of anemia and other blood disorder when employed as a hematopoietic substance, the eggshell polypeptides are optionally used in combination with cytokines; glycoprotein growth factors; colony-stimulating factors (CSFs) such as granulocyte-macrophage CSF (GM-CSF), granulocyte CSF (G-CSF) and macrophage CSF (M-CSF); erythropoietin; and combinations comprising one or more of the foregoing agents.

In one embodiment, a composition comprising eggshell polypeptides is employed to stimulate fibroblasts, for example, in the form of a topical composition. Fibroblasts, located in the dermal layer, produce components of the extracellular matrix like collagen and various cytokines, which, in turn, enhance the proliferation and migration of keratinocytes. Keratinocytes are located in the epidermal layer and form a barrier against the external environment. The compositions comprising eggshell polypeptides are useful in topical, e.g., cosmetic compositions for the treatment of skin barrier and cornification disorders, and for skin aging and/or wrinkle reduction. Eggshell polypeptides can be embedded in phosphatidylcholine liposomes or nanoparticles.

The component of eggshells that stimulates osteoblast formation is the polypeptide fraction of the eggshell, that is, a polypeptide extract from eggshells. As used herein, the term polypeptide means a plurality of amino acids joined by peptide bonds and includes proteins, protein fragments and peptides. An eggshell polypeptide is a polypeptide extracted from eggshells, or a fragment thereof. Preferably, the fragment is capable of stimulating osteoblast activity. Depending upon the technique utilized to extract the eggshell polypeptides, at least a portion of the polypeptides may be reduced in molecular mass compared to the polypeptides in their native state. Without being held to theory, it is believed that the relatively harsh procedures required to extract hard eggshell polypeptides result in a reduction in the average molecular weight of the isolated polypeptides, that it, at least a fraction of the polypeptides undergo some degradation during extraction.

The term hard eggshell tissue includes the calcified layers of eggshell, that is, the hard shell with the palisade and the mamillary layers. The term soft eggshell tissue includes the eggshell inner and outer eggshell membranes, also known as the membrane layers on the inner-side of the eggshells (within the egg).

In one embodiment, when processing (cleaning, sterilizing) the eggshells, they should remain as native as possible, that is, preserving the organic compounds (proteins, collagens) and not to denature them as is done with heat processing (e.g. autoclaving). Washing and sterilizing eggshells can be done by putting eggshells in water that is saturated with oxygen-ozone for 5-25 min. Surprisingly, the oxygenized/ozonized water did not denature the organic compound of the eggshells. After the washing with the ozonized water, the wet eggshells may be dried gently with a vacuum dryer.

In one embodiment, the hard eggshell polypeptides are extracted by contacting hard eggshell tissue with an aqueous reducing buffer comprising 1 to 25 wt % SDS. In another embodiment, the reducing buffer further comprises 1 to 60% vol/vol acetic acid. In another embodiment, the hard eggshell polypeptides are extracted using 1 to 60% vol/vol acetic acid solution. In one embodiment, the acetic acid solution further comprises 1 to 25 wt % EDTA.

In one embodiment, the hard eggshell extract comprises an Ovocleidin, an Ovocalyxin and/or a Clusterin. In another embodiment, the hard eggshell polypeptide extract comprises Ovocleidin-116, Clusterin-(sulfated glycoprotein 2), Ovocleidin-17, Ovocalyxin-32 active fragments thereof, or an active fraction comprising one or more of the foregoing. An active fraction is a fraction capable of stimulating osteoblast activity.

In one embodiment, the soft eggshell polypeptides are extracted by contacting soft eggshell tissue with a solution comprising 2 to 10 M urea and having a pH of 7.0-9.0. In one embodiment, the buffer comprises 25 mM Tris/HCl, pH 8.9, 2 M urea.

In one embodiment, the soft eggshell polypeptide extract comprises Ovoalbumin, Ovotransferrin precursor, 78 kDa polypeptide (Ovotransferrin family), 105 kDa polypeptide (Ovotransferrin family), Ovalbumin-related polypeptide Y, 52 kDa polypeptide (Ovoinhibitor Serine protease-inhibiting protein), Ovomucoid precursor, SERPINB11 similar to Ovalbumin-related protein Y, Ovoglycoprotein precursor, Lysozyme C precursor, 28 kDa polypeptide, Ovomucin alpha-subunit, Hep21 protein precursor, Ovocleidin-17, 164 kDa polypeptide, Clusterin 49 polypeptide, Ovostatin precursor, 18 kDa polypeptide, 8 kDa polypeptide, 35 kDa polypeptide, 34 kDa polypeptide, alpha-1 (type XI) collagen isoform, Isoform Alpha-KT of NT-3 growth factor receptor, Cystatin precursor, active fragments thereof, or an active fraction comprising one or more of the foregoing. An active fraction is a fraction capable of stimulating osteoblast activity.

In one embodiment, the hard eggshell polypeptide extract, the soft eggshell polypeptide extract, or both, is a total hard or soft eggshell polypeptide extract. By total extract it is meant that the extract comprises greater than 90 wt %, specifically greater than 95 wt %, and more specifically greater than 98 wt % of the weight of all polypeptides that can be isolated from the tissue. A total polypeptide extract may comprise a fraction of the polypeptides in degraded form so long as the total extract comprises the aforementioned amounts of polypeptide.

In one embodiment, rather than an eggshell polypeptide extract, a composition comprising purified recombinant eggshell polypeptides is employed. In one embodiment, the composition comprises a purified recombinant Ovocleidin and/or a purified recombinant Clusterin.

Polynucleotides suitable for the expression of eggshell polypeptides can be inserted into a recombinant expression vector or vectors. The term “recombinant expression vector” refers to a plasmid, virus, or other means known in the art that has been manipulated by insertion or incorporation of a genetic sequence. The term “plasmids” generally is designated herein by a lower case p preceded and/or followed by capital letters and/or numbers, in accordance with standard naming conventions that are familiar to those of skill in the art. Plasmids are either commercially available, publicly available on an unrestricted basis, or can be constructed from available plasmids by routine application of well-known, published procedures. Many plasmids and other cloning and expression vectors are well known and readily available, or those of ordinary skill in the art may readily construct any number of other plasmids suitable for use. These vectors may be transformed into a suitable host cell to form a host cell vector system for the production of a polypeptide.

In one embodiment, recombinant proteins are expressed using eukaryotic protein expression in Leishmania tarentolae.

The unicellular kinetoplast protozoan Leishmania tarentolae, isolated from the Moorish gecko Tarentola mauritanica, is not pathogenic to mammals (Biosafety level 1), and is the protein-producing host of the commercially available eukaryotic protein expression system LEXSY (Jena Bioscience).

The polynucleotides suitable for expression of eggshell polypeptides can be inserted into a vector adapted for expression in a bacterial, yeast, insect, amphibian, or mammalian, or other prokaryotic or eukaryotic cell, that further comprises the regulatory elements necessary for expression of the nucleic acid molecule in the bacterial, yeast, insect, amphibian, or mammalian cell operatively linked to the nucleic acid molecule encoding the polypeptide. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., atg) in front of a protein-encoding gene, splicing signals for introns (if introns are present), maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter. By “promoter” is meant minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included

Transformation of a host cell with an expression vector or other DNA may be carried out by conventional techniques as are well known to those skilled in the art. By “transformation” is meant a permanent or transient genetic change induced in a cell following incorporation of new DNA (i.e., DNA exogenous to the cell). Where the cell is a mammalian cell, a permanent genetic change is generally achieved by introduction of the DNA into the genome of the cell. By “transformed cell” or “host cell” is meant a cell (e.g., prokaryotic or eukaryotic) into which (or into an ancestor of which) has been introduced, by means of recombinant DNA techniques, a DNA molecule encoding an eggshell polypeptide of the invention, or fragment thereof.

The eggshell polypeptides can also be designed to provide additional sequences, such as, for example, the addition of coding sequences for added C-terminal or N-terminal amino acids that would facilitate purification by trapping on columns or use of antibodies. Such tags include, for example, histidine-rich tags that allow purification of polypeptides on Nickel columns. Such gene modification techniques and suitable additional sequences are well known in the molecular biology arts.

Eggshell proteins, polypeptides, or polypeptide derivatives can be purified by methods known in the art. These methods include, but are not limited to, size exclusion chromatography, ammonium sulfate fractionation, ion exchange chromatography, affinity chromatography, crystallization, electrofocusing, preparative gel electrophoresis, and combinations comprising one or more of the foregoing methods. A preparation of isolated and purified eggshell is about 50% to about 99.9% pure, with greater than or equal to about 80%, preferred, greater than or equal to about 85% purity more preferred, greater than or equal to about 90% purity more preferred, and greater than or equal to about 95% especially preferred. Purity may be assessed by means known in the art, such as SDS-polyacrylamide gel electrophoresis.

Compositions comprising eggshell polypeptides include, for example, solid, semi-solid and liquid dosage forms such as tablets, pills, powders, liquid solutions or suspensions, suppositories, and injectable and infusible solutions. The form depends on the intended mode of administration and therapeutic application and may be selected by one skilled in the art. Modes of administration include oral, parenteral, subcutaneous, intravenous, intralesional or topical administration. In one embodiment, the compositions are administered in the vicinity of the treatment site in need of bone or cartilage regeneration or repair.

In one embodiment, a composition comprising an eggshell polypeptide further comprises an excipient. Excipients may be added to facilitate manufacture, enhance stability, control release, enhance product characteristics, enhance bioavailability, enhance patient acceptability, and the like. Pharmaceutical excipients include binders, disintegrants, lubricants, glidants, compression aids, colors, sweeteners, preservatives, suspending agents, dispersing agents, film formers, flavors, printing inks, etc. Binders hold the ingredients in the dosage form together. Exemplary binders include polyvinyl pyrrolidone, hydroxypropyl cellulose, hydroxypropyl methylcellulose, methylcellulose and hydroxyethyl cellulose, and combinations comprising one or more of the foregoing binders. Disintegrants expand when wet causing a tablet to break apart. Exemplary disintegrants include water swellable substances, for example, low-substituted hydroxypropyl cellulose; cross-linked polyvinyl pyrrolidone; cross-linked sodium carboxymethylcellulose (sodium croscarmellose); sodium starch glycolate; sodium carboxymethylcellulose; sodium carboxymethyl starch; ion-exchange resins; microcrystalline cellulose; starches and pregelatinized starch; formalin-casein, and combinations comprising one or more of the foregoing water swellable substances. Lubricants, for example, aid in the processing of powder materials. Exemplary lubricants include calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants. Glidants include, for example, silicon dioxide.

In embodiments for topical applications, the carrier is in a form appropriate for topical application to the skin including, for example, solutions, colloidal dispersions, emulsions (oil-in-water or water-in-oil), suspensions, creams, lotions, gels, foams, mousses, sprays, shampoos and the like. Compositions suitable for use in topical application also include, for example, nanoparticle or liposomal carriers suspended in a suitable base or vehicle. A liquid, pharmaceutically acceptable vehicle in which the eggshell polypeptides are at least minimally soluble is suitable for topical use. Other preparations that may be suitable include application of the composition onto a polyvinyl alcohol sponge.

Dose and duration of therapy will depend on a variety of factors, including disease type, patient age, patient weight, and the like. Initial dose levels are selected based on their ability to achieve ambient concentrations shown to be effective in in-vitro models, in-vivo models and in clinical trials, up to maximum tolerated levels. The dose of a particular composition and duration of therapy for a particular patient can be determined by the skilled clinician using standard pharmacological approaches in view of the above factors. The response to treatment is monitored by analysis of blood or body fluid levels or levels in relevant tissues or monitoring disease state in the patient. The skilled clinician will adjust the dose and duration of therapy based on the response to treatment revealed by these measurements. In one embodiment, for oral administration, 5 to 1000 mg of eggshell polypeptides are administered, specifically 50 to 150 mg.

The invention is further illustrated by the following non-limiting examples.

### Example 1

### Eggshell Preparation

Commercially available fresh chicken eggs were purchased, opened, and the egg content removed by pouring. Then the eggshells were separated into two different tissues, the hard-tissue containing the calcified layers (hard shell with the palisade and the mamillary layers) and the soft-tissue containing the inner and outer egg shell membranes (membrane layers).

Several different polypeptide extraction procedures were used, four for the hard-tissue and one the soft-tissue of the eggshell.

### Example 2

### Hard Eggshell Tissue Polypeptide Extraction Procedure 1

The hard eggshell tissue was washed with water and pounded in a mortar, resulting in a white powder. The powder was dried overnight. Then, 1.77 g of the powder was dissolved in 700 μl of reducing SDS buffer (15% glycerol, 25 mM TRIS/HCl, pH 7.5, 2% SDS, 1% DTT) including protease inhibitors, and stirred for 1.5 hours. Further, 700 μl of reducing SDS-buffer were added and stirred for another hour. Then, the mixture was heated to 95° C. for 5 minutes, and subjected to an ultrasonication bath for 10 minutes. This procedure was repeated 3 times. Then, the sample was incubated overnight. The next day, the mixture was centrifuged at 13,000×g for 5 minutes. The supernatant was carefully removed and concentrated by 3 kDa ultrafiltration to 130 μl. To precipitate the polypeptides, the sample was diluted with 1.2 ml ice cold acetone and incubated overnight at 4° C. The precipitate was then dissolved in 50 μl 3-fold reducing SDS buffer and subjected to SDS-PAGE.

### Example 3

### Hard Eggshell Tissue Polypeptide Extraction Procedure 2

The hard eggshell tissue was washed with water and pounded in a mortar resulting in a white powder. The powder was dried overnight. Then, 1.0 g of this powder was dissolved in 300 μl of 3-fold reducing SDS-buffer and 300 ml of 10% acetic acid, and stirred overnight. The next day the mixture was centrifuged at 13,000×g for 5 minutes, and then 400 μl of 3-fold SDS reducing buffer were added and stirred for 30 minutes. Then, the mixture was heated to 95° C. for 5 minutes, subjected to an ultrasonication bath for 10 minutes, and centrifuged at 13,000×g for 5 minutes. The supernatant was carefully removed and concentrated by 3 kDa ultrafiltration to 20 μl. After another centrifugation step (13,000×g for 5 minutes), the supernatant was used for SDS-PAGE.

### Example 4

### Hard Eggshell Tissue Polypeptide Extraction Procedure 3

0.8 mg of hard eggshell tissue powder was dissolved in 700 μl of 5% EDTA and incubated under gentle agitation at 4° C. for 30 minutes. Then, 1300 μl of 10% acetic acid was added. The mixture was incubated overnight. The next day, the mixture was centrifuged at 14,000×g for 5 minutes. The supernatant was carefully removed and dialyzed 4×30 minutes against 250 ml of 5% sodium acetate buffer, pH 8.5. Then the solution was lyophilized resulting in a white powder.

### Example 5

### Hard Eggshell Tissue Polypeptide Extraction Procedure 4

The hard eggshell tissue was washed with water and pounded in a mortar resulting in a white powder. The powder was dried overnight. Then 100 g of this powder was dissolved in 35 ml of 50% acetic acid, and stirred over night. The next day, 50 ml of 50% acetic acid was added and stirred for further 4 hours. Then, 10 ml of water was added and incubated over night. The next day 10 ml of water was added, and the mixture was centrifuged at 14,000×g for 5 minutes. The supernatant was carefully removed and subjected to lyophilization resulting in a white powder.

### Example 6

### Soft Eggshell Tissue Polypeptide Extraction Procedure

The eggshell membranes (soft-tissue) were prepared from 2 fresh eggs and completely removed from the calcified layer. The eggshell membranes were extensively washed with PBS. Then they were frozen at −80° C. In the following protocol the two samples were kept separated but treated equally. They were pounded in a mortar but due to their paper-like consistency, scissors were taken instead to cut them in small pieces. They were then dissolved in 1.5 ml buffer (25 mM Tris/HCl, pH 8.9, 2 M urea). Glass beads were added, and the membranes were treated by an ultrasonifier (10×5 seconds). Then the mixtures were stirred at room temperature for 1 hour. This treatment was repeated 3 times. Then they were centrifuged at 13,000×g for 5 minutes. The supernatant was carefully removed and used for a Bradford assay as well as for SDS-PAGE. The supernatant was subject to lyophilization resulting in a pure protein powder.

### Example 7

### Analysis of Hard and Soft Eggshell Polypeptides by SDS-PAGE

The following techniques were used to analyze and identify polypeptides of the lyophilisate of both the hard eggshell tissue and soft eggshell tissue.

SDS-PAGE (Sodium dodecylsulfate-polyacrylamide gel electrophoresis): For separation of the eggshell polypeptides SDS-PAGE was used. The samples were diluted and heated in reducing sample buffer and were applied onto 4-20% SDS-PAGE gels (BioRad). The gels were then run for about 150 Vh. Some of the gels were then stained by Sypro Ruby, a highly sensitive fluorescent dye, scanned using a fluorescence scanner (Fuji FLA 3000), and restained by Coomassie Blue. Others were stained by silver.

The hard eggshell tissue was prepared according to different methods as described above, and applied to a SDS-PAGE, 4-20%, BioRad, and stained by silver. Only one gel showed the directly lyophilised polypeptides. The polypeptide concentration was measured by a Bradford assay before applying the samples on to the SDS-PAGE, which resulted in significant polypeptide amounts between 3-6 mg/ml.

For the hard eggshell polypeptide preparations, no clear polypeptide bands were visible on the SDS-PAGE gels using the four different preparations, although the Bradford assay indicated a significant polypeptide concentration. Without being held to theory, it is believed that the peptides that were present in the sample were too small to be detected on the SDS-PAGE.

In contrast to the hard eggshell polypeptides, the samples of the soft eggshell tissue (egg shell membranes) polypeptides revealed a nice distribution of extracted polypeptides.

### Example 8

### Analysis of Hard and Soft Eggshell Polypeptides by Nano-LC-ESI-MSMS

Nano-LC-ESI-MSMS: 1D-nano-LC separation was performed on a multi-dimensional liquid chromatography system (Ettan MDLC, GE Healthcare). Polypeptides were loaded on a RPC trap column with a flow-rate of 6 μl per minute (loading buffer: 0.1% formic acid; trap column: C18 PepMap 100, 5 μm bead size, 300 μm i.d., 5 mm length, LC Packings), and subsequently separated by an analytical column (C18 PepMap 100, 3 μm bead size, 75 μm i.d.; 15 cm length, LC Packings) with an 120-min linear gradient (A: 0.1% formic acid, B: 84% ACN and 0.1% formic acid) at a flow rate of 260 nl/min. The gradient was as follows: 0-30% B in 80 min., 30-60% B in 30 min., 60-100% B in 10 min.

Mass spectrometry was performed on a linear ion trap mass spectrometer (Thermo LTQ, Thermo Electron) online coupled to the nano-LC system. For electrospray ionization a distal coated SilicaTip (FS-360-50-15-D-20) and a needle voltage of 1.4 kV was used. The MS method consisted of a cycle combining one full MS scan (Mass range: 300-2000 m/z) with three data dependant MS/MS events (35% collision energy). The dynamic exclusion was set to 30 s.

10 μl of the eggshell soft tissue polypeptide extract was reduced, alkylated by iodacetamide, and digested by trypsin over night at 37° C. Then 10 μl of the sample were used for nano-LC-ESIMSMS runs. The MSMS spectra were then searched against IPI chicken plus Decoy-IPI chicken database using the MASCOT software. This has the advantage to see which hits are significant and when it becomes critical with the significance (in that moment when the first random hit appears). This procedure is much more accurate that just the Mascot scores which are dependent on the size of the data base, the number of modifications, and other factors.

MASCOT search result of the LC-ESI-MSMS run of the eggshell soft-tissue (eggshell membrane) polypeptides:
1. Ovalbumin SEQ ID NO:1 (gi|45383974|ref|NP_{—}990592.1)
2. Ovotransferrin SEQ ID NO:2 (gi|757851|emb|CAA26040.1|)
3. 78 KDa protein Histone-arginine N-methyltransferase PRMT7 SEQ ID NO:27 (gi|82233719|sp|Q5ZIB9.1)
4. 78 KDa protein Protein-glutamine gamma-glutamyltransferase SEQ ID NO:28 (gi|62903517|sp|Q01841.3)
5. 78 KDa protein Leprecan-like 1 SEQ ID NO:29 (gi|48374055|ref|NP_{—}001001530.1)
6. 105 KDa protein nebulin SEQ ID NO:20 (gi|1559266|dbj|BAB18735.1)
7. Ovoalbumin related protein Y SEQ ID NO: 4 (gi|118086485|ref|XP_{—}418984.2)
8. 52 KDa protein ovoinhibitor SEQ ID NO: 19 (gi|71895337|ref|NP_{—}001025783.1)
9. Ovomucoid SEQ ID NO:3 (gi|162952006|ref|NP_{—}001106132.1)
10. Ovoalbumin related protein SPARC (Secreted protein acidic and rich in cysteine or Osteonectin) SEQ ID NO: 32 (gi|548972|sp|P36377.1|)
11. Ovoglycoprotein SEQ ID NO:5 (gi|45383093|ref|NP_{—}989872.1)
12. Lysozyme C SEQ ID NO:6 (gi|45384212|ref|NP_{—}990612.1)
13. 28 kDA protein Apolipoprotein precursor SEQ ID NO: 33 (gi|211146|gb|AAA48592.1|)
   a. apolipoprotein SEQ ID NO: 34 (gi|211154|gb|AAA48595.1)
14. Ovomucin alpha SEQ ID NO:7 (gi|12583679|dbj|BAB21488.1)
15. Hep-21protein SEQ ID NO:8 (gi|45383131|ref|NP_{—}989852.1)
16. Ovocleidin 17 SEQ ID NO:9 (gi|1087046|gb|AAB35101.1)
   a. SEQ ID NO:10 (gi|32699622|sp|Q9PRS8.2)
   b. SEQ ID NO:11 (gi|31615312|pdb|1GZ2|A)
17. 164 KDa protein M-protein, striated muscle SEQ ID NO: 21 (gi|547919|sp|Q02173.1)
18. 49 KDa protein Clusterin SEQ ID NO:12 (gi|45382467|ref|NP_{—}990231.1)
19. Ovostatin SEQ ID NO:13 (gi|1683350gb|AH004621.1)
20. 18 KDa protein Insulin-like growth factor II SEQ ID NO:22 (gi|226693533|sp|P33717.2)
21. 8 KDa protein insulin-like growth factor 1 SEQ ID NO:23 (gi|52138671|ref|NP_{—}001004384.1)
22. *KDA protein fowlicidin-1 SEQ ID NO:24 (gi|242346718|gb|ACS92527.1)
23. 35 KDa protein WD repeat domain 82 pseudogene 1 SEQ ID NO:25 (gi|57524933|ref|NP_{—}001006135.1)
24. 34 KDa protein Sulfotransferase family cytosolic 1B member 1 SEQ ID NO:26 (gi|57013083|sp|Q8JG30.1)
25. Ovomucin beta SEQ ID NO:30 (gi|48147235|dbj|BAD22545.1)
26. NTF-3 protein (Neurotropin-3) SEQ ID NO:15 (gi|6175082|sp|Q91044.2)
27. Type XI collagen SEQ ID NO:14 (gi|63249|emb|CAA23688.1)
28. Cystatin SEQ ID NO:16 (gi|18195|sp|P01038.2)
29. Gallinacin-11 SEQ ID NO: 31 (gi|82173548|sp|Q6IV20.1)

### Hard Eggshell Polypeptides:

1. Ovocleidin-116 SEQ ID NO:18 (gi|45383041|ref|NP_{—}989900.1)
2. Clusterin 49 kDa SEQ ID NO:12 (gi|45382467|ref|NP_{—}990231.1)
   a. SEQ ID NO: 35 (gi|4325105|gb|AAD17257.1|)
3. Ovocleidin-17 SEQ ID NO:11 (gi|31615312|pdb|1GZ2)
4. Ovocalyxin-32 SEQ ID NO:17 (gi|78100756|sp|Q90YI1.1)

### Example 9

### Stimulation of Osteoblast Activity by Eggshell Polypeptides

The polypeptide lyophilisate was tested against autoclaved eggshells on osteoblasts (bone forming cells).

Eggshell powder was autoclaved (PO) at three different temperatures and/or periods of time. Additionally, eggshell polypeptides were extracted and isolated from fresh eggshells and lyophilized.

Six groups were formed for the cell culture studies:
1. Control (no additions)
2. PO-I (Autoclaved at 137° C.)
3. PO-II (Standard autoclaving at 121° C.)
4. HA−Hard Eggshell Polypeptides: 0.5 g/l [HA=Hyaluronic Acid]
5. Hard eggshell-Polypeptides (low concentration 0.005 g/l)
6. Hard eggshell-Polypeptides (high concentration 0.5 g/l)
7. PO-O (Autoclaved at 113° C.)

A 5-day proliferation study with osteoblasts was conducted for all five test groups and one control. After one and two weeks, an additional morphological comparison was carried out by light microscope. Further, after two weeks the expression of type I collagen, osteonectin, osteopontin and osteocalcin was qualitatively checked and evaluated.

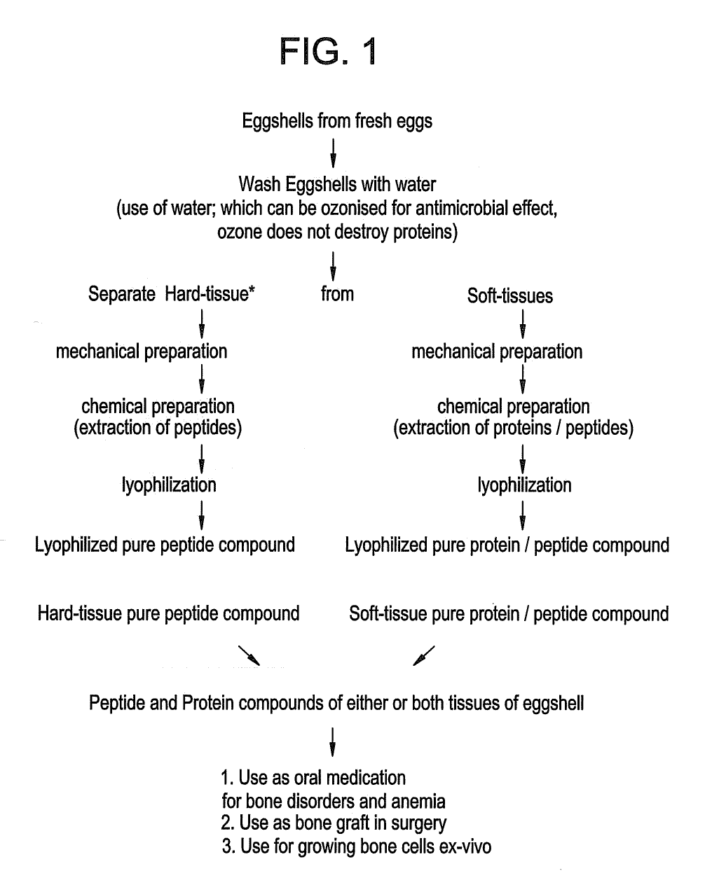

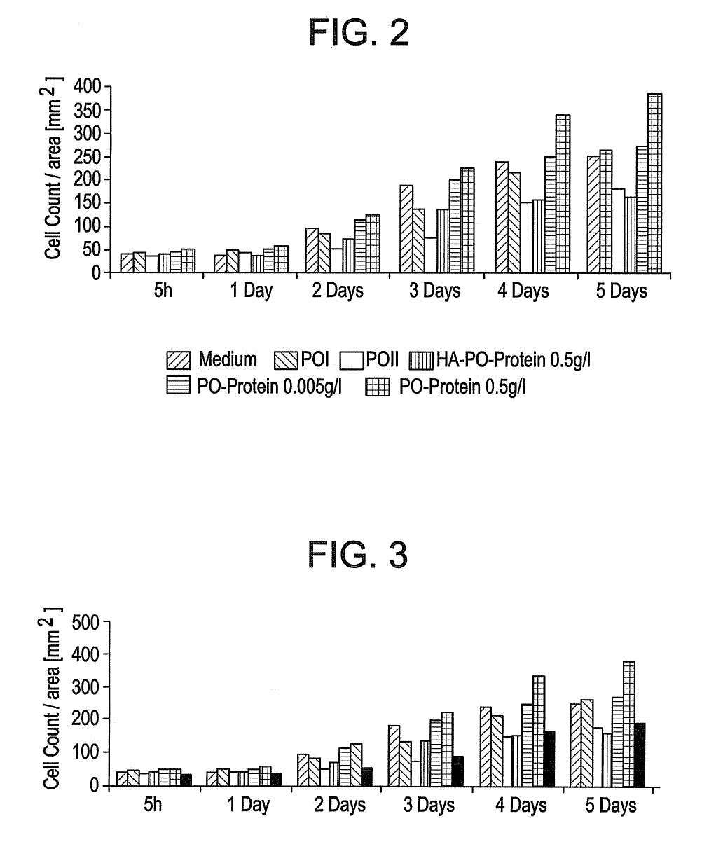

The cell count per screen was determined after 5 hours as well as after 1, 2, 3, 4 and 5 days to investigate the influence of the added substances on the proliferation of bovine osteoblasts. The daily count was done using a light microscope at 100× magnification. The results of the proliferation of the four-week old osteoblasts depending on the cultivation time are shown in FIGS. 1 and 2.

FIGS. 2 and 3 show a marked difference in the cell count increase between individual cell populations depending on the added substance. After a cultivation period of 5 hours, the bars for the additives PO Mod.I, PO Mod.II, HA+Hard eggshell polypeptides 0.5 g/l are within the range for the untreated culture medium. In contrast to this, a slightly increased cell count is observed for hard eggshell polypeptides 0.005 g/l and HA+hard eggshell polypeptides 0.5 g/l. On day 1, the bar for hard eggshell polypeptides 0.5 g/l compared with PO Mod. II, HA+hard eggshell polypeptides 0.5 g/l and the culture medium show significantly (p<0.05) increased cell counts. In contrast, there is only a tendency towards a higher cell count compared with hard eggshell polypeptides 0.005 g/l and PO Mod. I. Up to day 5, the cell counts of the cell cultures treated with hard eggshell polypeptides 0.5 g/l increased by a factor of 7. With the exception of the hard eggshell polypeptides 0.005 g/l, the cell count for the fresh hard eggshell polypeptides 0.5 g/l exceeds all the other samples significantly at times 2, 3, 4 and 5 days (p<0.001). The cell count in the untreated culture medium developed continually up to day 5. In the sample with added HA+hard eggshell polypeptides 0.5 g/l, only a minimal increase in the cell count is observed over the period day 3 to day 5.

In comparison with the two Putamen Ovi (PO) preparations, the cell count of the PO Mod. I is considerably higher than that of PO Mod. II. The means, standard deviations and significance levels of the substances investigated, PO Mod. I, POII Mod. II, hard eggshell polypeptides 0.005 g/l, hard eggshell polypeptides 0.5 g/l, HA+hard eggshell polypeptides 0.5 g/l and the culture medium. The cell culture treated with hard eggshell polypeptides 0.5 g/l serves as a reference for illustrating the statistical significance of the proliferation.

The test group PO-0 is conspicuously different to the rest. Although there is a tendency for the proliferation result to better than the control, it is poorer than the sample autoclaved at 137° C.

During the proliferation phase, the cells with the higher hard eggshell polypeptide concentrations and with the PO autoclaved at lower temperatures propagate and differentiate most strongly. Substitution with hyaluronic acid in combination with hard eggshell polypeptides and PO autoclaved at high temperatures did not cause any significant increase in cell proliferation or differentiation. The studies on the synthesis of bone-specific matrix proteins showed increased expression of type I collagen and osteopontin for the higher hard eggshell polypeptide concentration and the PO that was autoclaved at the lower temperature. Analysis of biomineralisation showed that the hard eggshell polypeptides both alone and in combination with hyaluronic acid led to considerably higher production of biomineralization and differentiation than any of the PO preparations.

The results show that the added substances PO, hard eggshell polypeptides and hyaluronic acid have a decisive influence on the factors of bone cell reaction. In particular, the high potential of fresh eggshell polypeptides has been shown for the first time.

It has been shown herein that eggshell polypeptides such as fresh eggshell polypeptides can stimulate bone cells to grow and activate their metabolism. Using eggshell polypeptides is a new mode of action to stimulate bone forming cells and to treat osteoporosis and other bone disorders. Other than bisphosphonates, which suppress osteoclasts, eggshell polypeptides stimulate osteoblasts significantly and stimulate osteoclasts slightly. This results in a higher metabolism level of both synergistic bone cells (osteoblasts and osteoclasts) with a higher impact on the osteoblasts. Another advantage is that there are no known side effects or adverse events related to administration of eggshell polypeptides. Bone advantageously grow faster when treated with fresh eggshell polypeptides than with bisphosphonates.

### Example 10

### Cell Proliferation Assay

In this example and those that follow, the following samples were employed:

SAMPLE A: Eggshell extracts from fresh eggs (various proteins) 0.5 g/L

SAMPLE B: Ovocleidin-16 and Ovocleidin-117 0.5 g/L

SAMPLE C: Clusterin (sulphated glycoprotein 2) and Ovocalyxin-32 0.5 g/L

Cell proliferation assays were done to investigate the influence of three different unknown protein-samples (A, B, C and B+C together, with no protein as control) on the cell proliferation of primary bovine osteoblast cells in vitro. For cell proliferation assays the bovine osteoblast like cells were cultured 4 weeks in MM1f-medium supplemented with the different protein samples A, B, C, B+C. Light-microscopy results of the osteoblast-like cells after 5 hours and 5 days cultured in the different media show that the number of cells increased from 5 hours to 5 days in culture independent from the medium/supplemented proteins employed. (data not shown) Cells were seeded sub-confluent and show their typical phenotype of more or less cuboidal cells after 5 hours in culture. After 5 days in culture cells nearly reached confluence and show in parts the beginning of their typical cobblestone-like appearance. Where the cells are still subconfluent with more space around the single cells, the size and shape of the osteoblast-like cells are not and phenotype varies from cuboidal/round to spindlelike.

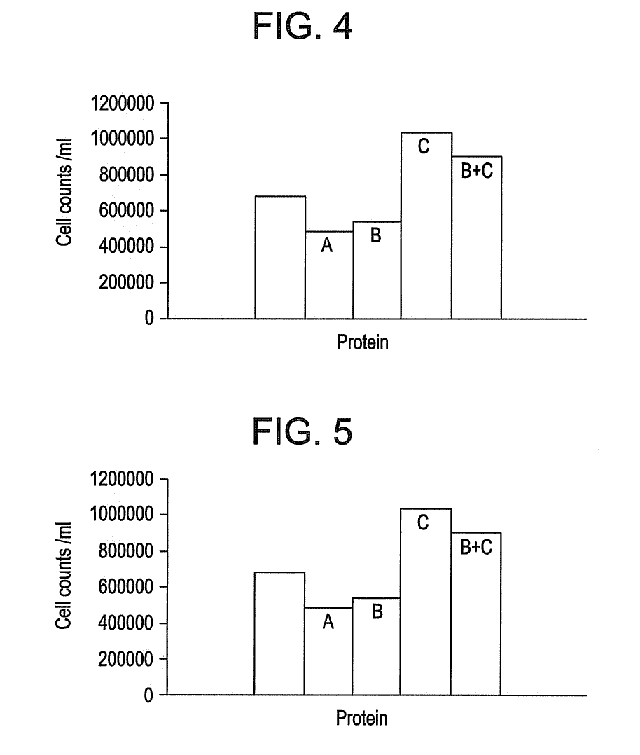

FIG. 4 shows the number of cells after 5 days in culture determined by the coulter counter system. Samples A and B show lower cell proliferation results (approximately 25%) as the control medium (MM1f) whereas samples C and B+C show higher cell numbers. The medium supplemented with protein specimen C yielded twice as many cells as the media with samples A or B. These results correspond also to the results of the other manual cell counting method.

In addition to the analysis of the cell number, the deviation of cell size is also detectable by the coulter counter system. FIG. 5 shows the medium cell sizes of the osteoblast cells after 5 days in culture with MM1f-medium as control and media supplemented with protein samples A, B, C, B+C.

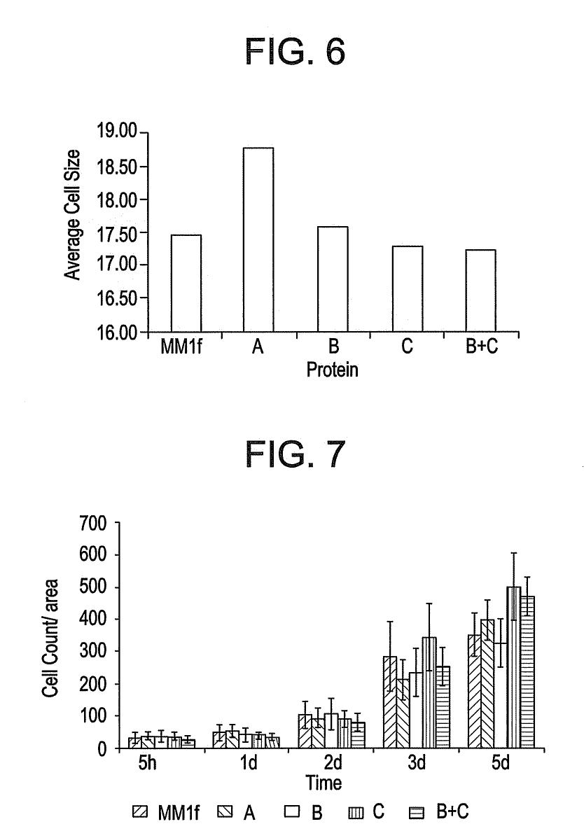

As shown in FIG. 6, the cell size of the primary osteoblast like cells after 5 days in culture is above 17 μm independent from the medium. But differences are also visible, medium with protein samples C and B+C yield cells with a lower cell size than the cells of the control (in MM1f-medium). Whereas the cell size of the osteoblast-like cells cultured in medium supplemented with protein specimen A with 18.77 μm is much higher than in MM1f. Protein sample B also seems to lead to an increase in cell size but smaller than protein sample A.

To investigate the influence of the supplemented protein specimens on the cell proliferation, the cells were cultured in the different media and number of cells was determined. Cells/image were calculated by counting the cells in a distinct area of an image using a light microscope with phase contrast after 5 h, 1 d, 2 d, 3 d, 4 d and 5 days in culture respectively. Analysis was done with the software program Image J. The mean values of the determined cell number (cells/image) over the culture time of 5 days are shown in FIG. 7. Standard deviations and p-values are listed below in Table 1.

The mean values of the cell numbers are all in the same range after 5 hours in culture. On day 1, the cell number increases in all used media. A small tendency to more cells within the supplemented media can be observed (p>0.05). The mean values of the counted cells in the supplemented media and the mean value of the cells in MM1f-medium after 5 hours in culture are more or less equal. After 1 day, the increase in cell number for the cells cultured in MM1f and in MM1f with sample A is somewhat higher than for the other media. The lowest increase is shown for medium with sample C, here the cell number increase is only 7% whereas the increase in MM1f is 32%. After 2 and 3 days all the media show high increase in cell number. The media with protein C (380%) and with samples B+C (320%) respectively show the highest values of cell number increase from day 2 to day 3. After 5 days, MM1f and medium with sample B show small increase in cell number. In contrast the cell number in medium supplemented with sample A show a higher increase, which cannot be supported by the results of the Coulter counter. The cell increase in the media with sample C and samples B+C respectively is significantly different from the MM1f-medium used as reference with high significance (p<0.01) for sample C and a most significant difference (p<0.001) for sample B+C.

TABLE 1
Overview of mean values, standard deviation and p-values of the
tested proteins A, B, C, B + C and MM1f medium as reference
concerning their influence on cell proliferation.

| | | | |
|---|---|---|---|
| | mean | standard | |
| time | value | deviation | p-value |

A

| | | | |
|---|---|---|---|
| 5 h | 39.67 | 12.06 | 0.328170441 |
| 1 d | 52.20 | 21.90 | 0.784840346 |
| 2 d | 90.11 | 34.19 | 0.23977764 |
| 3 d | 213.50 | 62.63 | 0.105811651 |
| 5 d | 398.90 | 64.14 | 0.141769379 |

B

| | | | |
|---|---|---|---|
| 5 h | 37.80 | 17.16 | 0.50898316 |
| 1 d | 41.50 | 20.20 | 0.46797194 |
| 2 d | 115.80 | 53.49 | 0.85477883 |
| 3 d | 235.20 | 75.17 | 0.27006054 |
| 5 d | 325.60 | 74.73 | 0.41740481 |

C

| | | | |
|---|---|---|---|
| 5 h | 37.10 | 12.56 | 0.519370985 |
| 1 d | 39.78 | 10.34 | 0.310625421 |
| 2 d | 91.22 | 26.44 | 0.241254906 |
| 3 d | 344.56 | 102.75 | 0.24192653 |
| 5 d | 500.50 | 98.57 | 0.001370099 |

B + C

| | | | |
|---|---|---|---|
| 5 h | 27.30 | 11.23 | 0.48406302 |
| 1 d | 35.20 | 11.35 | 0.14515393 |
| 2 d | 79.00 | 30.44 | 0.11073736 |
| 3 d | 254.10 | 57.79 | 0.46378238 |
| 5 d | 470.50 | 48.81 | 0.00055933 |

MM1f

| | | | |
|---|---|---|---|
| 5 h | 32.33333333 | 18.05547009 | x |
| 1 d | 49.22222222 | 24.57019423 | x |
| 2 d | 102.875 | 41.22217676 | x |
| 3 d | 284.4444444 | 107.0725351 | x |
| 5 d | 352.5555556 | 66.63353343 | x |

The values with the highest significance are marked red (p < 0.5 = significant, p < 0.01 = high significant, p < 0.001 = most significant)

Table 1 summarizes the mean values, standard deviations and the level of significance for the differently treated cells. The cells in MM1f-medium as control were taken as reference for statistical significance.

### Example 11

### Cell Differentiation

The cell layer was examined after immunohistochemistry and staining. (data not shown) The areas with the cells cultured in MM1f-medium or medium supplemented with sample B show a steady staining result. In contrast the cells cultured with the media supplemented with samples A or C or B+C show single and higher stained point shaped areas. Protein samples A and C were not completely in solution before use and the media were not filtered, so this can be a reason for the observed effect due to not solved but stained protein specimens.

The alizarin red staining (to detect calcium within the cells) shows, even by using light microscopy, only stained and not solved protein samples A, C and B+C, but no cell staining.

### Example 12

### Richardson-Staining

With the Richardson staining, basopile and osmophile structures of the cell can be visualised by a blue colour. By this method, the phenotypic characteristics like cell size and spreading can be easily analysed. (data not shown)

The bovine osteoblasts cultured in MM1f-medium show a uniform distribution, but the cell density is not as high as in the other media. The cell nuclei and a clear boundary from the extracellular matrix can be observed. The cells cultured in medium supplemented with protein sample A present blue dye in the cytoplasma as well as in the extracellular matrix. This applies also to the cells cultured in medium with sample B. The osteoblasts cultured in medium with sample C are bigger in size than in the other media. The form of some cells is more oval than roundish. The cells exhibit cell and the extracellular matrix is slightly dyed blue. The cells cultured in medium with sample B+C show clearly visible cell organelles and also a slight dyeing of the intercellular space.

### Example 13

### Collagen I

Collagen I is a fibril-forming protein and the main component of the extracellular matrix. All samples exhibit networks of Collagen I fibrils. (data not shown) In the case of sample A and sample B+C the fibrils seem to be both intra- and extracellular. The cells treated with sample B or C or with no supplement (MMf1) show less matrix structure and fibrils are located only extracellular. The cell sample cultured in MM1f medium presents the highest density of collagen fibrils, the results of the media with sample C and with samples B+C show more porous extracellular network with areas free of collagen fibrils.

### Example 14

### Osteocalcin

The immunohistochemical detection of osteocalcin shows a light staining of the cytoplasma of the cells cultured in MM1f-medium and those in medium with protein sample B and medium with sample C. (data not shown) In these cases no staining is visible on the extracellular side. In contrast the cells treated with protein sample A show osteocalcin staining in the cytoplasma and the surrounding extracellular matrix. In the sample of cells cultured in medium with sample B+C no osteocalcin is detectable.

### Example 15

### Osteonectin

After 2 weeks in culture the detection of osteonectin by immunohistochemistry of the MM1f-cultured cells show Osteonectin both intra- and extracellular. (data not shown) The medium with sample A results in highly stained areas and less stained areas determining the presence of osteonectin also in the intra- and the intercellular space. The cells cultured with sample B are highly stained intra- and extracellular, noticeable is the more elongated shape of the cells with cellular extensions. In contrast to these results the cells cultured with protein sample B+C show a determination of osteonectin only intracellular.

### Example 16

### Osteopontin

Ostopontin filaments can be seen in and out of the cells treated with non-supplemented medium (MM1f) and with medium supplemented with protein sample B. (data not shown) On the other hand some extracellular areas show only light or even no staining. In these areas nearly no cells are detectable. The result of the cells treated with sample C show ostonectin staining in the extracellular matrix and no dye in the cells' cytoplasma. In the sample with proteins B+C, no staining of the extracellular matrix is observed, but the cells are stained more or less uniform. A clear boundary between the cells and the intercellular space is visible.

### Example 18

### Biomineralisation of the Osteoblast-Like Cells

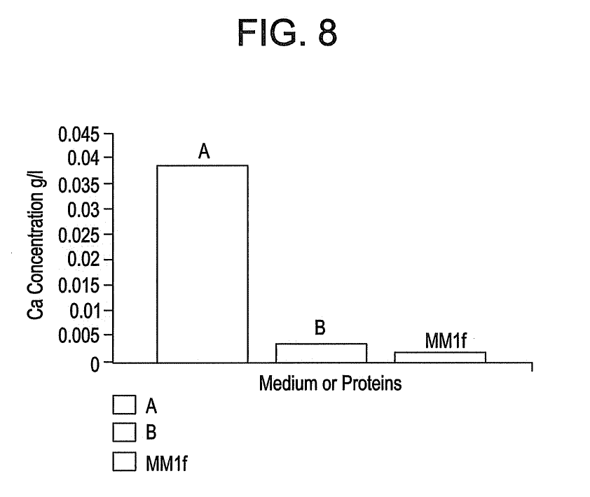

Spectral photometric analysis was done to investigate the biomineralization of the osteoblast-like cells by the quantification of the calcium concentration. The cells were cultured for 4 weeks with different media supplemented with the same protein specimens as before. MMf1-medium with no supplement was used as control. This experiment was done to examine the influence of the different protein specimens on the mineralization process. The calcium concentration was determined by a calibration line (y=mx+b) with y=8.1274x+0.0252. FIG. 8 shows the results of the calcium determination.

Protein sample A clearly shows a high impact (increasing metabolism) on osteoblasts performing biomineralization, which is essential for forming bone. Furthermore, sample A could produce highest rates of cell differentiation. Protein samples B and C have a stimulating effect on osteoblast cell proliferation but could not produce biomineralization as much as specimen A. Thus, the intact eggshell extract is more active than the individual components tested with regard to biomineralization.

The terms “a” and “an” herein do not denote a limitation of quantity, but rather denote the presence of at least one of the referenced item.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

All cited patents, patent applications, and other references are incorporated herein by reference in their entirety.

1. A method of stimulating osteoblast activity, comprising
contacting osteoblasts with a composition comprising a polypeptide extract from eggshells, wherein the polypeptide extract is isolated from a hard eggshell tissue, a soft eggshell tissue, or a combination thereof.

2. The method of claim 1, wherein the eggshells are fresh eggshells.

3. The method of claim 1, wherein the eggshells are from fertilized or unfertilized eggs.

4. The method of claim 1, wherein the polypeptide extract from hard eggshell tissue is isolated by treating hard eggshell tissue with a 1 to 25 wt % SDS and optionally 1 to 60% vol/vol acetic acid.

5. The method of claim 4, wherein the polypeptide extract from hard eggshell tissue comprises an Ovocleidin, an Ovocalyxin or a Clusterin.

6. The method of claim 1, wherein the polypeptide extract from soft eggshell tissue is isolated by treating soft eggshell tissue with a solution comprising 2 to 10 M urea and having a pH of 7.0-9.0.

7. The method of claim 6, wherein the polypeptide extract from soft eggshell tissue comprises Ovoalbumin, Ovotransferrin precursor, 78 kDa polypeptide (Ovotransferrin family), 105 kDa polypeptide (Ovotransferrin family), Ovalbumin-related polypeptide Y, 52 kDa polypeptide (Ovoinhibitor Serine protease-inhibiting protein), Ovomucoid precursor, SERPINB11 similar to Ovalbumin-related protein Y, Ovoglycoprotein precursor, Lysozyme C precursor, 28 kDa polypeptide, Ovomucin alpha-subunit, Hep21 protein precursor, Ovocleidin-17, 164 kDa polypeptide, Clusterin 49 polypeptide, Ovostatin precursor, 18 kDa polypeptide, 8 kDa polypeptide, 35 kDa polypeptide, 34 kDa polypeptide, alpha-1 (type XI) collagen isoform, Isoform Alpha-KT of NT-3 growth factor receptor, Cystatin precursor, SPARC polypeptide, active fragments thereof, or an active fraction comprising one or more of the foregoing

8. The method of claim 1,
wherein the osteoblasts are located at a site of injury in a mammalian subject, and wherein contacting comprises administering to the site of injury by injection.

9. The method of claim 1, wherein the polypeptide extract from eggshells is present at a concentration of 0.0001 g/L to 2 g/L.

10. The method of claim 1, wherein the polypeptide extract from eggshells is present at a concentration of 0.005 g/L to 0.5 g/L.

11. The method of claim 1, wherein the osteoblasts are present in ex-vivo culture.

12. The method of claim 1, wherein contacting osteoblasts comprises administering the composition to an individual in need of repair of cartilage.

13. The method of claim 1, wherein contacting osteoblasts comprises intraosseous injection or local application.

14. The method of claim 13, wherein the intraosseous injection or local application is for use in Kyphoplasty, Vertiboplasty, into fracture site, or into a bone or joint defect.

15. A method of stimulating osteogenic and/or osteoinductive activity in an individual in need thereof, comprising
administering to the individual a composition comprising a polypeptide extract from eggshells, wherein the polypeptide extract is isolated from a hard eggshell tissue, a soft eggshell tissue, or a combination thereof.

16. The method of claim 15 wherein the eggshells are fresh eggshells.

17. The method of claim 15, wherein the eggshells are from fertilized or unfertilized eggs.

18. The method of claim 15, wherein the polypeptide extract from hard eggshell tissue is isolated by treating hard eggshell tissue with a solution comprising 1 to 25 wt % SDS and optionally 1 to 60% vol/vol acetic acid.

19. The method of claim 18, wherein the polypeptide extract from hard eggshell tissue comprises an Ovocleidin, an Ovocalyxin or a Clusterin.

20. The method of claim 15, wherein the polypeptide extract from soft eggshell tissue is isolated by treating soft eggshell tissue with a solution comprising 2 to 10 M urea and having a pH of 7.0-9.0.

21. The method of claim 20, wherein the polypeptide extract from soft eggshell tissue comprises Ovoalbumin, Ovotransferrin precursor, 78 kDa polypeptide (Ovotransferrin family), 105 kDa polypeptide (Ovotransferrin family), Ovalbumin-related polypeptide Y, 52 kDa polypeptide (Ovoinhibitor Serine protease-inhibiting protein), Ovomucoid precursor, SERPINB11 similar to Ovalbumin-related protein Y, Ovoglycoprotein precursor, Lysozyme C precursor, 28 kDa polypeptide, Ovomucin alpha-subunit, Hep21 protein precursor, Ovocleidin-17, 164 kDa polypeptide, Clusterin 49 polypeptide, Ovostatin precursor, 18 kDa polypeptide, 8 kDa polypeptide, 35 kDa polypeptide, 34 kDa polypeptide, alpha-1 (type XI) collagen isoform, Isoform Alpha-KT of NT-3 growth factor receptor, Cystatin precursor, SPARC polypeptide active fragments thereof, or an active fraction comprising one or more of the foregoing.

22. The method of claim 15, wherein the individual is in need of treatment for a bone disorder.

23. The method of claim 22, wherein the bone disorder is osteoporosis, osteopenia, Osteogenesis Imperfecta, or Paget's disease, osteomalacia, osteopetrosis, Osgood-Schlatter disease, Algodystrophy, Reflex-Sympathetic-Dystrophy syndrome (Complex Regional Pain syndrome), transient osteoporosis, avascular necrosis, osteonecrosis, osteochondral lesions, osteolytic lesions, bone tumors, or bone fractures.

24. The method of claim 15, wherein administering comprises oral, intravenous, intramuscular, or nasal administration.

25. The method of claim 15, wherein the individual is in need of repair of cartilage.

26. A method of stimulating hematopoesis in an individual in need thereof, comprising
administering to the individual a composition comprising a polypeptide extract from eggshells, wherein the polypeptide extract is isolated from a hard eggshell tissue, a soft eggshell tissue, or a combination thereof.

27. The method of claim 26, wherein the individual is in need of treatment for anemia and related bone marrow and blood diseases.

28. A method of stimulating cartilage formation and/or differentiation in an individual in need thereof, comprising
administering to the individual a composition comprising a polypeptide extract from eggshells, wherein the polypeptide extract is isolated from a hard eggshell tissue, a soft eggshell tissue, or a combination thereof.

29. A method of stimulating fibroblasts in an individual in need thereof, comprising
administering to the individual a composition comprising a polypeptide extract from eggshells, wherein the polypeptide extract is isolated from a hard eggshell tissue, a soft eggshell tissue, or a combination thereof.

30. The product of the process of treating hard eggshell tissue with 1 to 25 wt % SDS and optionally 1 to 60% vol/vol acetic acid.

31. The product of claim 30, comprising an Ovocleidin, an Ovocalyxin or a Clusterin.

32. The product of the process of treating soft eggshell tissue with a solution comprising 2 to 10 M urea and having a pH of 7.0-9.0.


SEQUENCE LISTING (35 sequences)

SEQ ID NO: 1 - protein, 615 aa, Gallus gallus
MKWVTLISFIFLFSSATSRNLQRFARDAEHKSEIAHRYNDLKEETFKAVAMITFAQYLQRCSYEGLSKLVKDVVDLAQKCVANEDAPECSKPLPSIILDEICQVEKLRDSYGAMADCCSKADPERNECFLSFKVSQPDFVQPYQRPASDVICQEYQDNRVSFLGHFIYSVARRHPFLYAPAILSFAVDFEHALQSCCKESDVGACLDTKEIVMREKAKGVSVKQQYFCGILKQFGDRVFQARQLIYLSQKYPKAPFSEVSKFVHDSIGVHKECCEGDMVECMDDMARMMSNLCSQQDVFSGKIKDCCEKPIVERSQCIMEAEFDEKPADLPSLVEKYIEDKEVCKSFEAGHDAFMAEFVYEYSRRHPEFSIQLIMRIAKGYESLLEKCCKTDNPAECYANAQEQLNQHIKETQDVVKTNCDLLHDHGEADFLKSILIRYTKKMPQVPTDLLLETGKKMTTIGTKCCQLGEDRRMACSEGYLSIVIHDTCRKQETTPINDNVSQCCSQLYANRRPCFTAMGVDTKYVPPPFNPDMFSFDEKLCSAPAEEREVGQMKLLINLIKRKPQMTEEQIKTIADGFTAMVDKCCKQSDINTCFGEEGANLIVQSRATLGIGA

SEQ ID NO: 2 - protein, 705 aa, Gallus gallus
MKLILCTVLSLGIAAVCFAAPPKSVIRWCTISSPEEKKCNNLRDLTQQERISLTCVQKATYLDCIKAIANNEADAISLDGGQVFEAGLAPYKLKPIAAEIYEHTEGSTTSYYAVAVVKKGTEFTVNDLQGKNSCHTGLGRSAGWNIPIGTLLHWGAIEWEGIESGSVEQAVAKFFSASCVPGATIEQKLCRQCKGDPKTKCARNAPYSGYSGAFHCLKDGKGDVAFVKHTTVNENAPDLNDEYELLCLDGSRQPVDNYKTCNWARVAAHAVVARDDNKVEDIWSFLSKAQSDFGVDTKSDFHLFGPPGKKDPVLKDFLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPEKCVASSHEKYFGYTGALRCLVEKGDVAFIQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISNLKTCNPSDILQMCSFLEGK

SEQ ID NO: 3 - protein, 208 aa, Gallus gallus
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVDCSEYPKPDCTAEDRPLCGSDNKTYGNKCNFCNAVVESNGTLTLSHFGKC

SEQ ID NO: 4 - protein, 388 aa, Gallus gallus
MGSISAANAEFCFDVFNELKVQHTNENILYSPLSIIVALAMVYMGARGNTEYQMEKALHFDSIAGLGGSTQTKCGKSVNIHLLFKELLSDITASKANYSLRIANRLYAEKSRPILPIYLKCVKKLYRAGLETVNFKTASDQARQLINSWVEKQTEGQIKDLLVSSSTDLDTTLVLVNAIYFKGMWKTAFNAEDTREMPFHVTKEESKPVQMMCMNNSFNVATLPAEKMKILELPFASGDLSMLVLLPDEVSGLERIEKTINFEKLTEWTNPNTMEKRRVKVYLPQMKIEEKYNLTSVLMALGMTDLFIPSANLTGISSAESLKISQAVHGAFMELSEDGIEMAGSTGVIEDIKHSPELEQFRADHPFLFLIKHNPTNTIVYFGRYWSP

SEQ ID NO: 5 - protein, 203 aa, Gallus gallus
MLAFLVPVLILAMGLVGAHGTESPTCAPLVPADMDNATVDRLLGHWVYIMGASQYPPHMAEMRELKYATFTLFPGSHEDEFNVTEIMRLNETCVVKNSSKIHVFRHNSTLTHEDGQVVSMAELIHSDKDLFILKHFKDNHVGLSLSARTAEVTKEQLEEFEAQLRCHGFKLEEAFITSPKDACPAAGEETGEGSAATAEPQLG

SEQ ID NO: 6 - protein, 147 aa, Gallus gallus
MRSLLILVLCFLPLAALGKVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 7 - protein, 2108 aa, Gallus gallus
MEIKKERSFWIFCLIWSFCKGKEPVQIVQVSTVGRSECTTWGNFHFHTFDHVKFTFPGTCTYVFASHCNDSYQDFNIKIRRSDKNSHLIYFTVTTDGVILEVKETGITVNGNQIPLPFSLKSILIEDTCAYFQVTSKLGLTLKWNWADTLLLDLEETYKEKICGLCGNYDGNKKNDLILDGYKMHPRQFGNFHKVEDPSEKCPDVRPDDHTGRHPTEDDNRCSKYKKMCKKLLSRFGNCPKVVAFDDYVATCTEDMCNCVVNSSQSDLVSSCICSTLNQYSRDCVLSKGDPGEWRTKELCYQECPSNMEYMECGNSCADTCADPERSKICKAPCTDGCFCPPGTILDDLGGKKCVPRDSCPCMFQGKVYSSGGTYSTPCQNCTCKGGHWSCISLPCSGSCSIDGGFHIKTFDNKKFNFHGNCHYVLAKNTDDTFVVIGEIIQCGTSKTMTCLKNVLVTLGRTTIKICSCGSIYMNNFIVKLPVSKDGITIFRPSTFFIKILSSAGVQIRVQMKPVMQLSITVDHSYQNRTSGLCGNFNNIQTDDFRTATGAVEDSAAAFGNSWKTRASCFDVEDSFEDPCSNSVDKEKFAQHWCALLSNTSSTFAACHSVVDPSVYIKRCMYDTCNAEKSEVALCSVLSTYSRDCAAAGMTLKGWRQGICDPSEECPETMVYNYSVKYCNQSCRSLDEPDPLCKVQIAPMEGCGCPEGTYLNDEEECVTPDDCPCYYKGKIVQPGNSFQEDKLLCKCIQGRLDCIGETVLVKDCPAPMYYFNCSSAGPGAIGSECQKSCKTQDMHCYVTECVSGCMCPDGLVLDGSGGCIPKDQCPCVHGGHFYKPGETIRVDCNTCTCNKRQWNCTDNPCKGTCTVYGNGHYMSFDGEKFDFLGDCDYILAQDFCPNNMDAGTFRIVIQNNACGKSLSICSLKITLIFESSEIRLLEGRIQEIATDPGAEKNYKVDLRGGYIVIETTQGMSFMWDQKTTVVVHVTPSFQGKVCGLCGDFDGRSRNDFTTRGQSVEMSIQEFGNSWKITSTCSNINMTDLCADQPFKSALGQKHCSIIKSSVFEACHSKVNPIPYYESCVSDFCGCDSVGDCECFCTSVAAYARSCSTAGVCINWRTPAICPVFCDYYNPPDKHEWFYKPCGAPCLKTCRNPQGKCGNILYSLEGCYPECSPDKPYFDEERRECVSLPDCTSCNPEEKLCTEDSKDCLCCYNGKTYPLNETIYSQTEGTKCGNAFCGPNGMIIETFIPCSTLSVPAQEQLMQPVTSAPLLSTEATPCFCTDNGQLIQMGENVSLPMNISGHCAYSICNASCQIELIWAECKVVQTEALETCEPNSEACPPTAAPNATSLVPATALAPMSDCLGLIPPRKFNESWDFGNCQIATCLGEENNIKLSSITCPPQQLKLCVNGFPFMKHHDETGCCEVFECQCICSGWGNEHYVTFDGTYYHFKENCTYVLVELIQPSSEKFWIHIDNYYCGAADGAICSMSLLIFHSNSLVILTQAKEHGKGTNLVLFNDKKVVPDISKNGIRITSSGLYIIVEIPELEVYVSYSRLAFYIKLPFGKYYNNTMGLCGTCTNQKSDDARKRNGEVTDSFKEMALDWKAPVSTNRYCNPGISEPVKIENYQHCEPSELCKIIWNLTECHRVVPPQPYYEACVASRCSQQHPSTECQSMQTYAALCGLHGICVDWRGQTNGQCEATCARDQVYKPCGEAKRNTCFSREVIVDTLLSRNNTPVFVEGCYCPDGNILLNEHDGICVSVCGCTAQDGSVKKPREAWEHDCQYCTCDEETLNISCFPRPCAKSPPINCTKEGFVRKIKPRLDDPCCTETVCECDIKTCIINKTACDLGFQPVVAISEDGCCPIFSCIPKGVCVSEGVEFKPGAVVPKSSCEDCVCTDEQDAVTGTNRIQCVPVKCQTTCQQGFRYVEKEGQCCSQCQQVACVANFPFGSVTIEVGKSYKAPYDNCTQYTCTESGGQFSLTSTVKVCLPFEESNCVPGTVDVTSDGCCKTCIDLPHKCKRSMKEQYIVHKHCKSAAPVPVPFCEGTCSTYSVYSFENNEMEHKCICCHEKKSHVEKVELVCSEHKTLKFSYVHVDECGCVETKCPMRRT

SEQ ID NO: 8 - protein, 107 aa, Gallus gallus
MKLLFVGLALVLCVGVVEALQCKVCKYKIPYVGCFHGANETTCERRERCAIIKTSLGKVTLYYQQGCTSALNCGRERASDAESRLTSRYSCCETDLCNEKWDDDPTD

SEQ ID NO: 9 - protein, 24 aa, Gallus gallus
DPDGSGPGWVPTPGGSLGFFSREL

SEQ ID NO: 10 - protein, 142 aa, Gallus gallus
DPDGCGPGWVPTPGGCLGFFSRELSWSRAESFCRRWGPGSHLAAVRSAAELRLLAELLNASRGGDGSGEGADGRVWIGLHRPAGSRSWRWSDGTAPRFASWHRTAKARRGGRCAALRDEEAFTSWAARPCTERNAFVCKAAA

SEQ ID NO: 11 - protein, 142 aa, Gallus gallus
  misc_feature (61)..(61): Xaa can be any naturally occurring amino acid
DPDGCGPGWVPTPGGCLGFFSRELSWSRAESFCRRWGPGSHLAAVRSAAELRLLAELLNAXRGGDGSGEGADGRVWIGLHRPAGSRSWRWSDGTAPRFASWHRTAKARRGGRCAALRDEEAFTSWAARPCTERNAFVCKAAA

SEQ ID NO: 12 - protein, 448 aa, Gallus gallus
MALPLLALLSLGLVCQGGQGLVPPSELKQLSAAGSKYIDTEVENAINGVKQMKTLMDKTSKEHQAMLHTLEETKRRKEEAVKLALEKEKQLAEKQEVCNETMLSLWEECKPCLKHTCMRVYSKICHSGSGLVGRQLEELLNRSSPFSIWVNGERIDALLDREQRQERRFEDLEERFGLMEDGVEDIFQDSTQLYGPAFPFFRTPPFGGFREAFVPPVQRVRLVPPRRRLSRELHPFLQHPVHGFHRLFEMTQRMLDGGHGAWDHLLGGFESESRNFSTDRMVCREIRRNSAGCLRMRDECEKCREILAVDCSQTDPVQSQLREQFEDALRLAERFTRRYDDLLSAFQAEMLNTSSLLDQLNRQFGWVLRLGNLTQGTDGFLQVTTVFSKTPNLEDPSAPADTQVTVQLFDSEPLSLTVPGDISWDDPRFMEIVAEQALQHYKQNNTIE

SEQ ID NO: 13 - protein, 208 aa, Gallus gallus
ALTSGLGPDVYQFLQDMGMKFFTNSKIRQPTVCTRETVRPPSYFLNAGFTASTHHVKLSAEVAREERGKRHILETIREFFPETWIWDIILINSTGKASVSYTIPDTITEWKASAFCVLPPNVVEESARASVSVLGDILGSAMQNTQNLLQMPYGCGEQNMVLFAPNIYVLDYLNETQQLSEDMKSKTIGYLESGYQKQLSYKHPDGSY

SEQ ID NO: 14 - protein, 461 aa, Gallus gallus
GARGPSGPVGSPGPNGAPGEAGRDGNPGNDGPPGRDGAPGFKGERGAPGNPGPSGALGAPGPHGQVGPSGKPGNRGDPGPVGPVGPAGAFGPRGLAGPQGPRGEKGEPGDKGHRGLPGLKGHNGLQGLPGLAGQHGDQGPPGNNGPAGPRGPPGPSGPPGKDGRNGLPGPIGPAGVRGSHGSQGPAGPPGPPGPPGPPGPNGGGYEVGFDAEYYRADQPSLRPKDYEVDATLKTLNNQIETLLTPEGSKKNPARTCRDLRLSHPEWSSGFYWIDPNQGCTADAIRAYCDFATGETCIHASLEDIPTKTWYVSKNPKDKKHIWFGETINGGTQFEYNGEGVTTKDMATQLAFMRLLANHASQNITYHCKNSIAYMDEETGNLKKAVILQGSNDVELRAEGNSRFTFSVLVDGCSKKNNKWGKTIIEYRTNKPSRLPILDIAPLDIGGADQEFGLHIGPVCFK

SEQ ID NO: 15 - protein, 827 aa, Gallus gallus
MDVSLCPTKCTFWRVFLLWSIWGDYLLSVLACPANCLCSKTDINCKKPDDGNLFPLLEGQDSGSSNGNTSINITDISRNITSIHIENWKNLQTLNAVDMELYTGLQRLTIRNSGLRNIQPRAFAKNPHLRYIDLSGNRLTTLSWQLFQTLRLFDLRLERNPFNCSCDIRWIQLWQEKGEANLQSQQLHCMNLDTAVILLRNMNITQCDLPEISVSHVNLTVREGENAVITCNGSGSPLPDVDWTVADLHSINTHQTNLNWTNVHAINLTLVNVTSEDNGFLLTCIAENVVGMSNASVLLTVYYPPRILTLEEPVLHLEHCIAFAVHGNPAPTLHWLHNGQVLRETEIIHMEFYQQGEVSEGCLLFNKPTHYNNGNYTIVATNQLGSANQTIKGHFLEKPFPESTDNFVSIGDYEVSPTPPITVTHKPEEDTFGVSIAVGLAAFACVLLVVLFIMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTNDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRVGGHTMLPIRWMPPESIMYRKFTTESDVWSFGVILWEIFTYGKQPWFQLSNTEVIECITQGRVLERPRVCPKEVYDIMLGCWQREPQQRLNIKEIYKILHALGKATPIYLDILG

SEQ ID NO: 16 - protein, 139 aa, Gallus gallus
MAGARGCVVLLAAALMLVGAVLGSEDRSRLLGAPVPVDENDEGLQRALQFAMAEYNRASNDKYSSRVVRVISAKRQLVSGIKYILQVEIGRTTCPKSSGDLQSCEFHDEPEMAKYTTCTFVVYSIPWLNQIKLLESKCQ

SEQ ID NO: 17 - protein, 275 aa, Gallus gallus
MPGLRAALPAALLLLSSFPPAAAERLPWPQVPGVMRPLNPSHREAVWAAWTALHYINSHEASPSRPLALHKVVKAASKMIPRLGWKYYVHCTTEGYIHGENAGSCFATVLYLKKSPPVVHGKCVHAQNKKQIQEEDHRFYEYLQHQKKPITANYIPDSNGNIAHDHLQLWGLAIVGSSYIMWKQSTEHTGYLLAQVSSVKQQIRKDNAVAFKFIVLLHEIPTQQLNVCHMYLVWTLGHPIRVKYSCAPDNHGLEDGSGQDSGSAAGTSHETKGNF

SEQ ID NO: 18 - protein, 743 aa, Gallus gallus
MRATLFCLCLCLLGTVLPTPVSLPARARGNCPGQHQILLKGCNTKHGFYIFQYIYSHLMQKNQTQVKKEEGDHQGTIHGHWLGKVDGEAPGQGVGSSHVPEDKDSPKPHSHITPASKGEGRALRPGIGDSNSVYPTSTSVEGSGDMGSILLGEIINGEDGLPHSTHPGGPHGDGDGGNGVLVDGAVTAGRERASGSKGAGSEGGSHATVPDQGQAGTMGTGDSAITSVTDSAITSVTKKEDVHVDTEGIDEFAYIPDVDAVTITRGQDGETHISPEDEVKIFIGRANIQVGENDSSVGSAGATSEANVIPTVVTVRPQGHPEESATMATLHHGDSVTSRPVGHPSVGNSGDGATEIHSGQELEAPSPWESTGGDATVTMAVGVQSGKGRSGQRALGKHSLPATMTTRGGRGTASSGLTTGDCSTAASTPSRKGSHVVTAGQGESGEVGTAGPERQRSRVQQEVAPARGVVGGMVVPEGHRARVQQEVAPSRGVVGGMVVPEGHRARTQPEVASAPSTVGKAAPERHRNRAQQEVAPVPSMVVETVAPERHRARVRPESARLGQAARPEVAPAPSTGGRIVAPGGHRARVWPGAAPAPGVVGVARPAPSKAYNGDKRVAIGKSTDVPRDPWVWGSAHPQAQHTRGSTVAGGFAHLHRGQRLGGLTEMEHSRQVEQVRHADRLRLHERAVYGLSGVGGPLQPPAVHTDPWSADSSQSSEGRWGSHSDSREEDGEVRGYPYGRQSL

SEQ ID NO: 19 - protein, 472 aa, Gallus gallus
MRTARQFVQVALALCCFADIAFGIEVNCSLYASGIGKDGTSWVACPRNLKPVCGTDGSTYSNECGICLYNREHGANVEKEYDGECRPKHVMIDCSPYLQVVRDGNTMVACPRILKPVCGSDSFTYDNECGICAYNAEHHTNISKLHDGECKLEIGSVDCSKYPSTVSKDGRTLVACPRILSPVCGTDGFTYDNECGICAHNAEQRTHVSKKHDGKCRQEIPEIDCDQYPTRKTTGGKLLVRCPRILLPVCGTDGFTYDNECGICAHNAQHGTEVKKSHDGRCKERSTPLDCTQYLSNTQNGEAITACPFILQEVCGTDGVTYSNDCSLCAHNIELGTSVAKKHDGRCREEVPELDCSKYKTSTLKDGRQVVACTMIYDPVCATNGVTYASECTLCAHNLEQRTNLGKRKNGRCEEDITKEHCREFQKVSPICTMEYVPHCGSDGVTYSNRCFFCNAYVQSNRTLNLVSMAAC

SEQ ID NO: 20 - protein, 904 aa, Gallus gallus
ETMNKRLYTEAWNKDKTTIHVMPDTPEILLAKQNQAHYSQKMYKLALEESKKKGHDLRFDAIPIQAAKASREIASDYKYKEGYRKQLGHHIGARNIEDDPKMMWSMHVAKIQSDREYKKAFEKTKTHFSSPVDMLGIVLAKKCQELVSDVDYRHYLHQWICLPDQNDVIHARKAYDLQSDAVYKSDLEWLKGIGWVPIGSLDVEKAKKAGEILSDRKYRQPADQIKFTSVTDSLAMMLAKHNAEIMNKRLYTEAWDADKTSIHVMPDTPTILLAKANAANVSHKHYVQAWEDAKAKNYDLRADAIPIKHAKASRDIASEYKYKEAHEKQKGHYIGCRTAKEDPKLSWAARAMLLQNDRIYRKAYNDSKAHIHMPVDAMSLQAAKECQTLVSDVDYRHYLHQWTCLPDHNDVVHARKAYDLQSDNVYKSDLEWLRGIGWLTEGSVDVVKAKKAQEILSDRLYRTQPDKMKFTSVTDAPDVVQAKINAMQLSNHLYREAWDKDKTQISIPSDTPEMLQSKVNALNISNKHYQKAWDEAKAKNYDLRADAIPIKHAKASRDIASEYKYKEAHEKQKGHYIGCRTAKEDPKLSWAARAMLLQNDRIYRKAYNDSKAHIHMPVDAMSLQAAKECQTLVSDVDYRHYLHQWTCHPDQNDCIQARKAYDLQSDNIYKSDLEWLRGCGWIPLGSVEHKKVKHAQELINKRAYTKDAIENFSKYTSVVDTPDIVLAKINSVNQSDLKYKETFNLEKGQYIGSDDTPELNHARDMSLLYSDKLYKRDWEVCKPIGYTLDAKYIPLVGAKHANYVNSELKYKEIYEKLKGHYLAGKDIGDFPSVVHSLAFQKIRSALAYRKNYEDTKTRVHIPSDMMNHVLAKKCQYILSDLEYRTYLHHWNCSPEEHDVIQARR

SEQ ID NO: 21 - protein, 1450 aa, Gallus gallus
MSSVAVPFYQRRHKHFDQSYRNIQTRYVLEEYAARKAASRQAAHYESTGLGKTTCRLCARRARSLAHEAMQESRKRTHEQKSHASDEKRIKFASELSSLEREIHMARHHAREQLDRLAIQRMVEENMALERHVVEEKISRAPEILVRLRSHTVWEKMSVRLCFTVQGFPSPVVQWYKNEELITPASDPAKYSVENKYGVHVLHINRADFDDSATYSAVATNIHGQASTNCAVVVRRFRESEEPHPAGIMPFHLPLSYDVCFTHFDVQFLEKFGVTFATEGETLTLKCSVLVTPELKRLRPRAEWYRDDVLIKDSKWTKLYFGEGQAALSFTHLNKDDEGLYTLRMVTKGGVNECSAFLFVRDADALIAGAPGAPMDVKCHDANRDYVIVTWKPPNTTSQNPVIGYFVDKCEVGLENWVQCNDAPVKICKYPVTGLYEGRSYIFRVRAVNSAGISRPSRVSEPVAALDPVDLERTQTVHVDEGRKIVISKDDLEGDIQIPGPPTNVHASEISKTYVVLSWDPPVPRGREPLTYFIEKSMVGSGSWQRVNAQVAVKSPRYAVFDLAEGKPYVFRVLSANKHGISDPSEITEPIQPQDIVVVPSAPGRVVATRNTKTSVVVQWDKPKHEENLYGYYIDYSVVGSNQWEPANHKPINYNRFVVHGLETGEQYIFRVKAVNAVGFSENSQESEAIKVQAALTCPSYPHGITLLNCDGHSMTLGWKAPKYSGGSPILGYYIDKREANHKNWHEVNSSVISRTIYTVEDLTEDAFYEFKIAAANVVGIGHPSDPSEHFKCKAWTMPEPGPAYDLTVCEVRNTSLVLLWKAPVYEGKSPITGYLVDYKEVDTEDWITANEKPTSHRYFKVTDLHQGHTYVFKVRAVNDAGVGKSSEISEPVFVEASPGTKEIFSGVDEEGNIYLGFECKEATDASHFLWGKSYEEIEDSDKFKIETKGDHSKLYFKHPDKSDLGTYCISVSDTDGVSSSFVLDEEELERLMTLSNEIKNPTIPLKSELAYEVLDKGEVRFWIQAESLSPNSTYRFVINDKEVENGDRHKISCDHSNGIIEMVMDKFTIDNEGTYTVQIQDGKAKNQSSLVLIGDAFKAILAESELQRKEFLRKQGPHFSEFLYWEVTEECEVLLACKIANTKKETVFKWYRNGSGIDVDEAPDLQKGECHLTVPKLSRKDEGVYKATLSDDRGHDVSTLELSGKVYNDIILALSRVSGKTASPLKILCTEEGIRLQCFLKYYNEEMKVTWSHRESKISSGEKMKIGGGEDVAWLQITEPTEKDKGNYTFEIFSDKESFKRTLDLSGQAFDDALTEFQRLKAAAFAEKNRGKVIGGLPDVVTIMDGKTLNLTCTVFGNPDPEVVWFKNDKALELNEHYLVSLEQGKYASLTIKGVTSEDSGKYSIYVKNKYGGETVDVTVSVYRHGEKIPEVNQGQLAKPRLIPPSSST

SEQ ID NO: 22 - protein, 187 aa, Gallus gallus
MCAARQILLLLLAFLAYALDSAAAYGTAETLCGGELVDTLQFVCGDRGFYFSRPVGRNNRRINRGIVEECCFRSCDLALLETYCAKSVKSERDLSATSLAGLPALNKESFQKPSHAKYSKYNVWQKKSSQRLQREVPGILRARRYRWQAEGLQAAEEARAMHRPLISLPSQRPPAPRASPEATGPQE

SEQ ID NO: 23 - protein, 153 aa, Gallus gallus
MEKINSLSTQLVKCCFCDFLKVKMHTVSYIHFFYLGLCLLTLTSSAAAGPETLCGAELVDALQFVCGDRGFYFSKPTGYGSSSRRLHHKGIVDECCFQSCDLRRLEMYCAPIKPPKSARSVRAQRHTDMPKAQKEVHLKNTSRGNTGNRNYRM

SEQ ID NO: 24 - protein, 79 aa, Gallus gallus
MLSCWVLLLALLGGACALPAPLGYSQALAQAVDSYNQRPEVQNAFRLLSADPEPGPNVQLSSLHNLNFTIMETRCQARS

SEQ ID NO: 25 - protein, 313 aa, Gallus gallus
MKLTDNVLRSFRVAKVFRENSDKINCFDFSPNGETVISSSDDDSIVLYDCQEGKPKRTLYSKKYGVDLIRYTHAANTVVYSSNKIDDTIRYLSLHDNKYIRYFPGHTKRVVALSMSPVDDTFISGSLDKTIRLWDLRSPNCQGLMHLQGKPVCSFDPEGLIFAAGVNSEMVKLYDLRSFDKGPFATFKMQYDRTCEWTGLKFSNDGKLILISTNGGFIRLIDAFKGAVLHTFGGYNNSKAVTLEASFTPDSQFIMIGSEDGKIHVWNGESGMKVAVLDGKHTGPITCLQFNPKFMTFASACSNMAFWLPTIDD

SEQ ID NO: 26 - protein, 296 aa, Gallus gallus
MGTVDAYLRQPWSTVHAIPMVSAFAQNWERVDNFQSRPDDIVVATFPKSGTTWISEIVDMILQGGDPKKCKRDAIVNRVPMLEFAAPGQMPAGTEQLENMPSPRIIKTHIPADILPKSFWDKSCKMIYVGRNAKDVAVSYYHFDLMNKLHPHPGTWDQYLEAFMAGKVAYGSWFDHVRGYWERRQEHPILYLFYEDMKEDLRREVAKVAQFLGRELTEVALDAIAHHTSFEAMRDNPSTNYSVVPSQLMDHGISPFMRKGITGDWKNHFTVAQSAHFDQYYAQKMAGTDLRFRTHI

SEQ ID NO: 27 - protein, 689 aa, Gallus gallus
MKTFCGRANPTTGSLEWVEEDEDYDYHQEIARSRYADMLHDKDRNMKYYQGIRAAVSRVKGRGEKAIVLDIGTGTGLLSMMAASAGADFCYAVEVFKPMANAAVKIVEKNGFGDKIKVINKHSTEVTVGPDGDMQCRANILVTELFDTELIGEGALPTYEHAHKYLVQEGCEAVPHRATVYVQLVESKRMWSWNKLFPVHVEAEDGEKIIVSPSEMENCPGVPSVCDIQLNQMPSSDFTILSDVVTMFSVDFSKPVRSASTCYRAQLDPVKSGKAQIVLSWWDIDMDPSGTINCTMAPYWVKPMSAFQWRDHWMQCVYFLPKEEQVLQGEKVHLTACRDEYSVWYTLQKAREEDESKADARVESPVCRCQAHLLWNRPRFGELNDQNRTRQYIKSLMSVLRTDSVCLCISDGSLLPVLAHYLGAEQVFTLENSAVSCSVMKKFFKANHLEDKIKIVEARPELLTSSHLEEKKISVLVGEPFFTTSLLPWHNLYFWYARTAVTEHLASDVTVLPQSAALHMMIVEFQDLWRIRSPCGTCEGFDVQTMDDMIKNSLNFRESKEAEPHPLWEYPCKSLSNPQEVLLFDFRKTVPQHCLSTEGSVNLLRKGKSHGAVLWMEYHLTADISVSTGLMQISNEKGNCEWNPHCKQAVYFFSSVIESETLADVPTAVTYAIKFDTKTGEIAMDFKLL

SEQ ID NO: 28 - protein, 689 aa, Gallus gallus
MAEELVLETCDLQCERNGREHRTEEMGSQQLVVRRGQPFTITLNFAGRGYEEGVDKLAFDVETGPCPVETSGTRSHFTLTDCPEEGTWSAVLQQQDGATLCVSLCSPSIARVGRYRLTLEASTGYQGSSFHLGDFVLLFNAWHPEDAVYLKEEDERREYVLSQQGLIYMGSRDYITSTPWNFGQFEDEILAICLEMLDINPKFLRDQNLDCSRRNDPVYIGRVVSAMVNCNDEDHGVLLGRWDNHYEDGMSPMAWIGSVDILKRWRRLGCQPVKYGQCWVFAAVACTVMRCLGVPSRVVTNYNSAHDTNGNLVIDRYLSETGMEERRSTDMIWNFHCWVECWMTRPDLAPGYDGWQALDPTPQEKSEGVYCCGPAPVKAIKEGDLQVQYDIPFVFAEVNADVVYWIVQSDGEKKKSTHSSVVGKNISTKSVGRDSREDITHTYKYPEGSEKEREVFSKAEHEKSSLGEQEEGLHMRIKLSEGANNGSDFDVFAFISNDTDKERECRLRLCARTASYNGEVGPQCGFKDLLNLSLQPHMEQSVPLRILYEQYGPNLTQDNMIKVVALLTEYETGDSVVAIRDVYIQNPEIKIRILGEPMQERKLVAEIRLVNPLAEPLNNCIFVVEGAGLTEGQRIEELEDPVEPQAEAKFRMEFVPRQAGLHKLMVDFESDKLTGVKGYRNVIIAPLPK

SEQ ID NO: 29 - protein, 694 aa, Gallus gallus
MAPGSRSWGAVLLLAAMLPAACGSCGADGGPLEPFDALYASGVEAYYGGDFAGAARCLEQALRSRRELRAERLRCRRRCRGQVRLAALGAGPAGELPFFGALLRRAGCLRSCEEPRLGAASRHRAAEEVRSDFQRRVPYSYLQRAYIQLNKLEEAANAAHTFFMANPEHMEIQQDIENYKTTAGKVSLIDLEAKPHMEDYSAGVRHYDKEEYGLAITFLERALEGYYAEDEDCQIMCEGPQRFEEHEYLEYKAGLYEAIADHYMQVLACKHDCIRELATRSGRISPIENFLPLHYDYLQFAYYRVGDYVKALECARSYLLFHPDDEDVLENAAYYEGLLEGTVDPATIKPRKEAKALLRRHKLESHLLRVAAVGLGFTYTEPNYWKRYGARQDEHSVPSSISSEPEDGPRLSLTKKPTPKPDRELKEGGPLLYSDVKFVYNSQQLNGTQRVLLDNVISEEQCRELHRVASGIMLAGDGYRGKTSPHTPNERFEGATVLKALKYGYEGRVPLKSARLFYDISEKARRIVESYFMLNSTLYFSYTHLVCRTALSGQQERRNDLSHPIHADNCLLDPEANECWKEPPAYTFRDYSALLYMNADFEGGEFIFTEMDAKTVTASIKPKCGRMVSFSSGGENPHGVKAVTKGQRCAVALWFTLDPLYRELERIQADEVIAMLDQEHVGRSEMNINPKDEL

SEQ ID NO: 30 - protein, 827 aa, Gallus gallus
PTRPYDFTGTCNYIFATVCDESSPDFNIQFRRGLDKKIARIIIELGPSVIIVEKDSISVRSVGVIKLPYASNGIQIAPYGRSVRLVAKLMEMELVVMWNNEDYLMVLTEKKYMGKTCGMCGNYDGYELNDFVSEGKLLDTYKFAALQKMDDPSEICLSEEISIPAIPHKKYAVICSQLLNLVSPTCSVPKDGFVTRCQLDMQDCSEPGQKNCTCSTLSEYSRQCAMSHQVVFNWRTENFCSVGKCSANQIYEECGSPCIKTCSNPEYSCSSHCTYGCFCPEGTVLDDISKNRTCVHLEQCPCTLNGETYAPGDTMKAACRTCKCTMGQWNCKELPCPGRCSLEGGSFVTTFDSRSYRFHGVCTYILMKSSSLPHNGTLMAIYEKSGYSHSETSLSAIIYLSTKDKIVISQNELLTDDDELKRLPYKSGDITIFKQSSMFIQMHTEFGLELVVQTSPVFQAYVKVSAQFQGRTLGLCGNYNGDTTDDFMTSMDITEGTASLFVDSWRAGNCLPAMERETDPCALSQLNKISAETHCSILTKKGTVFETCHAVVNPTPFYKRCVYQACNYEETFPYICSALGSYARTCSSMGLILENWRNSMDNCTITCTGNQTFSYNTQACERTCLSLSNPTLECHPTDIPIEGCNCPKGMYLNHKNECVRKSHCPCYLEDRKYILPDQSTMTGGITCYCVNGRLSCTGKLQNPAESCKAPKKYISCSDSLENKYGATCAPTCQMLATGIECIPTKCESGCVCADGLYENLDGRCVPPEECPCEYGGLSYGKGEQIQTECEICTCRKGKWKCVQKSRCSSTCNLYGEGHITTFDGQRF

SEQ ID NO: 31 - protein, 104 aa, Gallus gallus
MKLFSCLMALLLFLLQAVPGLGLPRDTSRCVGYHGYCIRSKVCPKPFAAFGTCSWRQKTCCVDTTSDFHTCQDKGGHCVSPKIRCLEEQLGLCPLKRWTCCKEI

SEQ ID NO: 32 - protein, 298 aa, Gallus gallus
MRTWIFFFLCLAGKALAAPQEALADETEVIEDLTTEGPVGANPVQVEVGEFEEPTEDVEEIVAENPCQNHHCKHGKVCEVDDNNSPMCVCQDPSSCPAHSGVFEKVCGTDNKTYDSSCHFFATKCTLEGTKKGHKLHLDYIGPCKFIPACLDTELTEFPLRMRDWLKNVLITLYERDEDNNLLTEKQKLKVKNIHENEKRLEAGDHTVELLARDFEKNYNMYIFPVHWQFGQLDQHPIDGYLSHTELAPLRAPLIPMEHCTTRFFEACDLDFDKYIALEEWASCFGIKEQDIDKDLVI

SEQ ID NO: 33 - protein, 264 aa, Gallus gallus
MRGVLVTLAVLFLTGIQARSFWQHDEPQTPLDRIRDMVDVYLETVKASGKDAIAQFESSAVGKQLDLKLADNLDTLSAAAAKLREDMAPYYKEVREMWLKDTEALRAELTKDLEEVKEKIRPFLDQFSAKWTEELEQYRQRLTPVAQKLKELTKQKVELMQAKLTPVAEEARDRLRGHVEELRKNLAPYSDELRQKLSQKLEEIREKGIPQASEYQAKVMEQLSNLREKMTPLVQEFRERLTPYAENLKNRLISFLDELQKSVA

SEQ ID NO: 34 - protein, 433 aa, Gallus gallus
IPGLSEKYTGEELYLMTTEKAAKTADICLSKLQEYFDALIAAISELEVRVPASETILRGRNVLDQIKEMLKHLQEKIRQTFVTLQEADFAGKLNRLKQVVQKTFQKAGNMVRSLQSKNFEDIKVQMQQLYKDAMASDYAHKLRSLAENVKKYISQIKNFSQKTLQKLSENLQQLVLYIKALREEYFDPTTLGWSVKYYEVEDKVLGLLKNLMDTLVIWYNEYAKDLSDLVTRLTDQVRELVENYRQEYYDLITDVEGKGRQKVMELSSAAQEKIRYWSAVAKRKINEHNRQVKAKLQEIYGQLSDSQEKLINVAKMLIDLTVEKYSTFMKYIFELLRWFEQATADSIKPYIAVREGELRIDVPFDWEYINQMPQKSREALRNKVELTRALIQQGVEQGTRKWEEMQAFIDEQLATEQLSFQQIVENIQKRMKT

SEQ ID NO: 35 - protein, 448 aa, Gallus gallus; identical to SEQ ID NO: 12